(19) United States
(12) Patent Application Publication
(10) Pub. No.: US 2026/0272616 A1
(43) Pub. Date: Sep. 17, 2026

(54) APPARATUS AND METHOD FOR RAPID HANDS-FREE APPLICATION OF GLOVES

(71) Applicant: Jeremy Lane Thompson, Eagle Mountain, UT (US)

(72) Inventor: Jeremy Lane Thompson, Eagle Mountain, UT (US)

(21) Appl. No.: 19/674,022

(22) Filed: May 11, 2026

Related U.S. Application Data
(63) Continuation of application No. 19/397,793, filed on Nov. 21, 2025.
Application No. 19/397,793 is a continuation of application No. 18/131,876, filed on Apr. 7, 2023, now abandoned.
Application No. 18/131,876 is a continuation-in-part of application No. 17/990,697, filed on Nov. 20, 2022.

(51) Int. Cl. A61C 8/00

(52) U.S. Cl. CPC A61C 8/0089; A61C 8/0066; A61C 8/0068

(57) ABSTRACT

A glove-donning apparatus includes a support structure and a pair of rings attached to the support structure. Each ring defines an opening configured to receive a hand of a user and to support an opening of an elastic glove stretched thereover. In certain embodiments, the rings are non-continuous and define a gap to enable a user's hand or arm to pass through the rings. The rings may be positioned to receive both hands of the user simultaneously, and the gloves may be positioned such that thumbs of the gloves are directed toward one another. When a user inserts his or her hands into the gloves and moves the hands through the rings, the gloves disengage from the rings and contract around the user's hands. The apparatus may enable the user to don gloves without handling the gloves during the donning process.

### BACKGROUND

### Field of the Invention

This invention relates to dentistry, and more particularly to apparatus and methods for securing full-arch shell temporaries, crowns, brides, dentures, or other prosthetic dental devices within a patient's mouth.

### Background of the Invention

Full-arch dentistry refers to the branch of dentistry that replaces a patient's natural teeth with full arches of prosthetic teeth. This may be performed for the upper and/or lower arches of the patient's teeth. This is typically accomplished by placing implants into the bone of the upper and/or lower arches of the patient's mouth and then attaching full arches of prosthetic teeth to these implants, typically with screws. Shortly after implants are placed in the patient's mouth but prior to attaching permanent prosthetic teeth to the implants, temporary arches of prosthetic teeth may be attached to the implants to allow the patient's gums and bone time to heal from the implant placement surgery, and to see if the patient likes the size, color, feel, and/or performance (e.g., talking, chewing, etc.) of the temporary arches.

Currently, implants may be placed using a surgical guide that attaches to a patient's existing teeth and/or gums to provide a guide for drilling holes for the implants. However, these surgical guides may have some significant limitations. For example, the surgical guides may obscure the dentist's view of the patient's mouth and make it difficult for the dentist to feel the bone into which the holes are being drilled, potentially causing non-optimal or unsatisfactory placement of the implants. The surgical guides also typically need to be made in a laboratory before the dentist can proceed with the implant-placement surgery. This adds significant time, expense, and patient visits to perform the full-arch procedure.

In addition, the temporary arches that are attached to the implants also typically need to be outsourced to a laboratory that is located remotely from the dentist's office. These temporary arches are also expensive and may take significant time to get back from the laboratory. In short, using current techniques, replacing a patient's natural teeth with full arches of prosthetic teeth typically requires a significant amount of pre-planning and outsourcing to a laboratory. This requires significant time, expense, and additional patient visits. If any of the temporary arches or surgical guides are not correct, the laboratory may need to be called upon again to make necessary corrections. Even with the added time and expense, current techniques may also result in a unsatisfactory or non-optimal end product, with the potential for non-optimal or unsatisfactory placement of the implants and temporary arches that are not optimal or satisfactory in terms of size, color, feel, and performance.

### SUMMARY

The invention has been developed in response to the present state of the art and, in particular, in response to the problems and needs in the art that have not yet been fully solved by currently available apparatus and methods. Accordingly, apparatus and methods for facilitating rapid donning of gloves are disclosed. The features and advantages of various embodiments of the invention will become more fully apparent from the following description and appended claims, or may be learned by practice of the invention as set forth hereinafter.

Consistent with the foregoing, a glove-donning apparatus is disclosed. In one embodiment, such an apparatus includes a support structure and a pair of rings attached to the support structure. Each ring defines an opening configured to receive a hand of a user and to support an opening of an elastic glove stretched thereover. In certain embodiments, the rings are non-continuous and define a gap to enable a user's hand or arm to pass through the rings. The rings may be positioned to receive both hands of the user simultaneously and, in certain embodiments, the gloves may be positioned such that thumbs of the gloves are directed toward one another. When a user inserts his or her hands into the gloves and moves the hands through the rings, the gloves may disengage from the rings and contract around the user's hands. In certain embodiments, the apparatus is configured such that the user may don the gloves without handling the gloves during the donning process.

A corresponding method is also disclosed and claimed herein.

### BRIEF DESCRIPTION OF THE DRAWINGS

In order that the advantages of the invention will be readily understood, a more particular description of the invention briefly described above will be rendered by reference to specific embodiments illustrated in the appended drawings. Understanding that these drawings depict only typical embodiments of the invention and are not therefore to be considered limiting of its scope, the embodiments of the invention will be described and explained with additional specificity and detail through use of the accompanying drawings, in which:

FIG. 1 shows various views of an upper arch of a full-arch shell temporary in accordance with the invention;

FIG. 2 shows various views of a lower arch of a full-arch shell temporary in accordance with the invention;

FIG. 3 is a front view of the full-arch shell temporary showing occlusion between the upper arch and the lower arch;

FIG. 4 is a front view of the upper arch of the full-arch shell temporary showing one embodiment of a nasal spine saddle that may be integrated therewith;

FIG. 5 shows grooves that may be incorporated into bases of the hamular notch and nasal spine saddles to facilitating breaking off the saddles when they are no longer needed;

FIG. 6 shows various views of one embodiment of a hamulometer in accordance with the invention;

FIG. 7 shows various views of one embodiment of a jig device for determining locations of implants in a patient;

FIG. 8 shows various views of one embodiment of a shell guide in accordance with the invention, in particular an upper arch of the shell guide;

FIG. 9 shows various views of one embodiment of a lower arch of the shell guide illustrated in FIG. 8;

FIG. 10 shows various views of one embodiment of an adjustable-angle multi-unit abutment in accordance with the invention;

FIGS. 11 and 12 shows various views of a cylinder that may be attached to an adjustable-angle multi-unit abutment in accordance with the invention;

FIG. 13 is a cross-sectional view of one embodiment of an abutment incorporating a snap cap in accordance with the invention;

FIG. 14 shows various views of another embodiment of an abutment incorporating a snap cap in accordance with the invention;

FIG. 15 shows various views of another embodiment of an adjustable-angle multi-unit abutment incorporating a snap cap in accordance with the invention, including skirts that may be used to keep reline material off of the abutment;

FIG. 16 is a process flow diagram showing one embodiment of a method for fitting a patient with an upper arch of a full-arch shell temporary in accordance with the invention;

FIG. 17 is a continuation of the process flow diagram of FIG. 16;

FIG. 18 is a process flow diagram showing one embodiment of a method for fitting a patient with a lower arch of a full-arch shell temporary in accordance with the invention;

FIG. 19 is a process flow diagram showing various alternative steps usable with the method of FIGS. 16 and 17, wherein the full-arch shell temporary is outfitted with snap caps in accordance with the invention;

FIG. 20 is a perspective view of one embodiment of a glove holder in accordance with the invention;

FIG. 21 is a perspective view of the glove holder of FIG. 20 with a pair of gloves fitted thereon;

FIG. 22 shows various views of another embodiment of a cylinder, for attachment to various types of prosthetic dental devices, that may be attached to an adjustable-angle multi-unit abutment in accordance with the invention;

FIG. 23 shows cross-sectional views of the cylinder and adjustable-angle multi-unit abutment of FIG. 22;

FIG. 24 shows various views of another embodiment of a cylinder that may be attached to an adjustable-angle multi-unit abutment in accordance with the invention;

FIG. 25 shows various views of yet another embodiment of a cylinder that may be attached to an adjustable-angle multi-unit abutment in accordance with the invention;

FIG. 26 shows various views of another embodiment of an adjustable-angle multi-unit abutment incorporating a snap cap, a skirt that may be used to keep reline material off of the abutment, and a rounded screw with a skirt to keep a patient's tissue away from the abutment;

FIG. 27 shows various views of another embodiment of an adjustable-angle multi-unit abutment in accordance with the invention, where the adjustable-angle multi-unit abutment is in an angled position;

FIG. 28 shows various views of the adjustable-angle multi-unit abutment of FIG. 27 in a straight position;

FIG. 29 shows various views of one embodiment of a driver in accordance with the invention;

FIG. 30 shows several views of a paralleling jig in accordance with the invention;

FIG. 31 is a perspective view showing the paralleling jig used with two adjustable-angle multi-unit abutments;

FIG. 32 is a perspective view of another embodiment of a paralleling jig that may be used with adjustable-angle multi-unit abutments having different heights;

FIG. 33 shows various views of another embodiment of a hamulometer in accordance with the invention;

FIG. 34 shows various views of yet another embodiment of a hamulometer in accordance with the invention;

FIG. 35 shows various views of a driver assembly that may be used within the mouth of a patient;

FIG. 36 shows various views of the driver assembly of FIG. 35 and further showing a finger wheel that may be used with the driver assembly;

FIG. 37 shows various enlarged views of the driver component used with the driver assembly of FIG. 35;

FIG. 38 shows various views of one embodiment of a dental implant that may be used in narrow sections of bone;

FIG. 39 shows various views of one embodiment of a finger-mounted tool holder for tools such as dental burs or drill bits;

FIG. 40 shows various views of a profiler that may be used to create a desired profile in a patient's bone for an abutment;

FIG. 41 shows various views of one embodiment of a ratcheting finger wheel with variable torque adjustment;

FIG. 42 shows one embodiment of an osteotome in accordance with the invention;

FIG. 43 shows one embodiment of an inverted adjustable-angle multi-unit abutment in accordance with the invention; and

FIG. 44 shows one embodiment of an inverted adjustable-angle multi-unit abutment interfacing with a wider-style dental implant.

### DETAILED DESCRIPTION

It will be readily understood that the components of the present invention, as generally described and illustrated in the Figures herein, could be arranged and designed in a wide variety of different configurations. Thus, the following more detailed description of the embodiments of the invention, as represented in the Figures, is not intended to limit the scope of the invention, as claimed, but is merely representative of certain examples of presently contemplated embodiments in accordance with the invention. The presently described embodiments will be best understood by reference to the drawings, wherein like parts are designated by like numerals throughout.

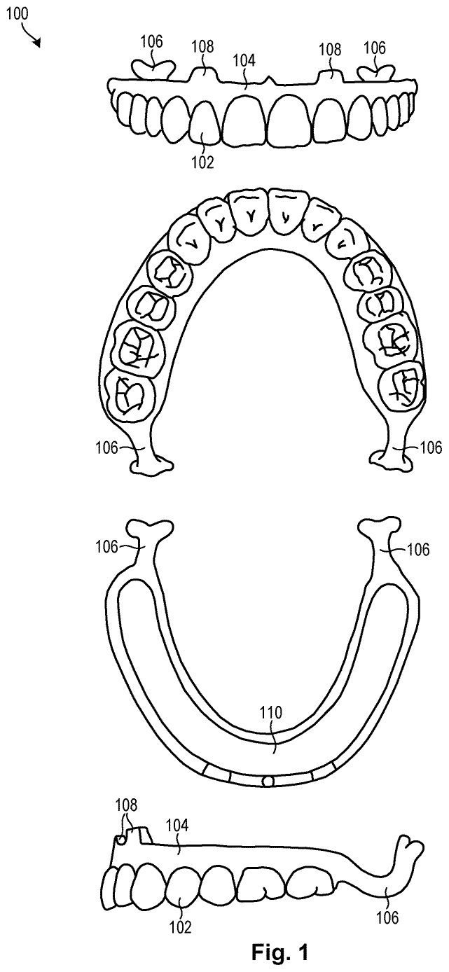

Referring to FIG. 1, as previously described, full-arch dentistry is a branch of dentistry that replaces a patient's natural teeth with full arches of prosthetic teeth. This is typically accomplished by placing implants into the bone of the upper and/or lower arches of the patient's mouth and then attaching arches of prosthetic teeth to these implants, typically with screws. Shortly after implants are placed in the patient's mouth but prior to attaching permanent prosthetic teeth to the implants, temporary arches of prosthetic teeth may be attached to the implants to allow the patient's gums and bone time to heal from the implant placement surgery, and to see if the patient likes the size, color, feel, and/or performance (e.g., talking, chewing, etc.) of the temporary arches.

Currently, implants may be placed using a surgical guide that attaches to a patient's existing teeth and/or gums to provide a guide for drilling holes for the implants. However, these surgical guides may have significant limitations. For example, the surgical guides may obscure the dentist's view of the patient's mouth and make it difficult for the dentist to feel the bone into which the holes are being drilled, potentially causing non-optimal or unsatisfactory placement of the implants. In some cases work may need to be repeated if the placement is incorrect or unsatisfactory. The surgical guides also typically need to be made in a laboratory before the dentist can proceed with the implant-placement surgery. This adds significant time, expense, and patient visits to perform the full-arch procedure.

In addition, the temporary arches that are attached to the implants also typically need to be outsourced to a laboratory that is located remotely from the dentist's office. These temporary arches are expensive and may take significant time to get back from the laboratory. In short, using current techniques, replacing a patient's natural teeth with full arches of prosthetic teeth typically requires a significant amount of pre-planning and outsourcing to a laboratory. This requires significant time, expense, and additional patient visits. If any of the temporary arches or surgical guides are not correct, the laboratory may need to be called upon again to make necessary corrections. Even with the added time and expense, current techniques may not result in a satisfactory or optimal end product, with the potential for non-optimal or unsatisfactory placement of the implants and temporary arches that are not optimal or satisfactory in terms of size, color, feel, and performance.

In view of the shortcomings disclosed above, alternative products and techniques are needed for fitting a patient with temporary arches of prosthetic teeth. Ideally, such alternative products and techniques will significantly speed up the process for fitting a patient with temporary prosthetic teeth, significantly reduce costs and the need to pre-plan, and provide the dentist more control over the process as well as the ability to customize and make adjustments to the end product in his or her office without the need for a laboratory. Ideally, such products and techniques will result in more optimally placed implants, as well as temporary prosthetic teeth and eventual permanent prosthetic teeth that are more optimal in terms of size, color, feel, and performance.

FIG. 1 shows various views of an upper arch 100 of temporary prosthetic teeth (hereinafter referred to as a “full-arch shell temporary”) in accordance with the invention, and more particularly, a front view, bottom view, top view, and side view of the upper arch 100. As shown, the upper arch 100 may include a first portion 102 that is made to resemble and function as a person's teeth and a second portion 104 that is made to resemble and function as a person's gums. As further shown in FIG. 1, in certain embodiments, the top side 110 of the upper arch 100 may be hollowed out to enable the upper arch 100 to fit over any protruding bone/gums, implant abutments, and the like when placing the upper arch 100 in a patient's mouth. The full-arch shell temporary may be made of acrylic resin or other materials known in the art of full-arch dentistry. In certain embodiments, the full-arch shell temporary is 3-D printed although the full-arch shell temporary is not limited to any particular method of manufacture.

One of the difficulties in full-arch dentistry is that a patient who may be a candidate for prosthetic teeth may arrive at a dentist's office, possibly with few if any teeth or teeth that can be preserved, and thus with few reference points from which to place a new set of prosthetic teeth. Nevertheless, there are various anatomical landmarks in the patient's mouth that may never change and may provide reference points from which to correctly place prosthetic teeth. For example, in certain embodiments, hamular notches in the patient's mouth may provide reference points that may be used to assist in placing new prosthetic teeth in the patient's mouth in a way that is not only aesthetically pleasing but also anatomically correct.

Thus, in certain embodiments, an upper arch 100 of a full-arch shell temporary may, in certain embodiments, be designed to enable accurate placement of the full-arch shell temporary relative to the hamular notches of a patient. For example, in one embodiment, the upper arch 100 may be equipped with temporary saddles 106 (also referred to herein as “hamular notch saddles 106”) to engage the hamular notches of a patient. These temporary saddles 106, when engaged with the patient's hamular notches, may enable the upper arch 100 to pivot with respect to the hamular notches. As shown, the temporary saddles 106 have a “Y” shape where the top of the “Y” is configured to engage the hamular notches.

Once the pivots points are established, an angle may be selected for the upper arch 100 that is ideally both aesthetically pleasing and anatomically correct in the patient's mouth. To find this angle, the upper arch 100 may include one or more reference members. For example, in certain embodiments, reference tabs 108 may be provided on the gum portion 104 of the upper arch 100 for alignment with a mucogingival junction (i.e., the junction between the soft, fleshy mucus membrane of the oral cavity and the tough, collagen rich gingiva) of the patient. The reference tabs 108 may be sized and positioned on the upper arch 100 such that aligning these elements with the mucogingival junction may achieve an angle that is both aesthetically pleasing and anatomically correct.

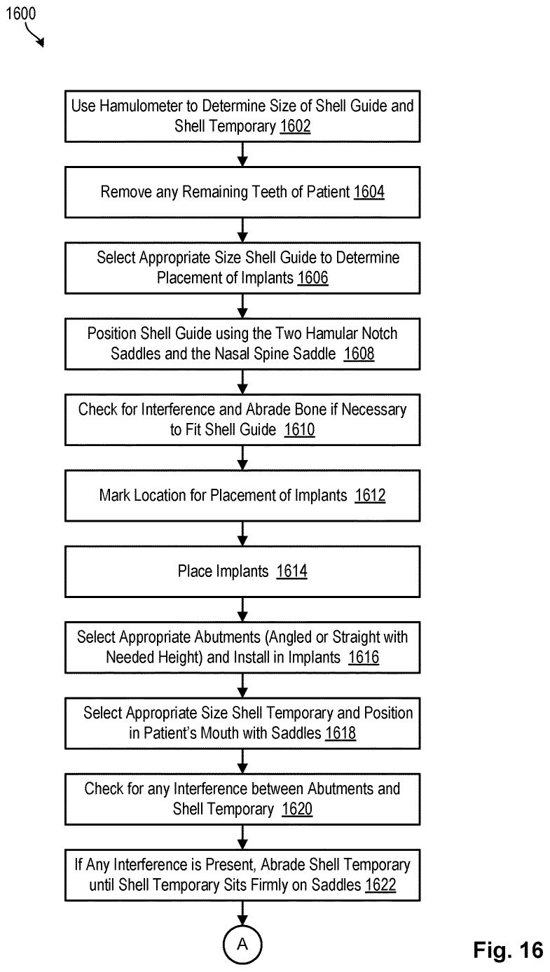

Once a desired position is determined for the upper arch 100 in the patient's mouth, the temporary saddles 106 may be removed from the upper arch 100, such as by snapping, cutting, or grinding the temporary saddles 106 off. The upper arch 100 may then be fixed in the patient's mouth, as will be explained in more detail hereafter. A more detailed explanation of how this may be accomplished will be discussed in association with FIGS. 16 through 19.

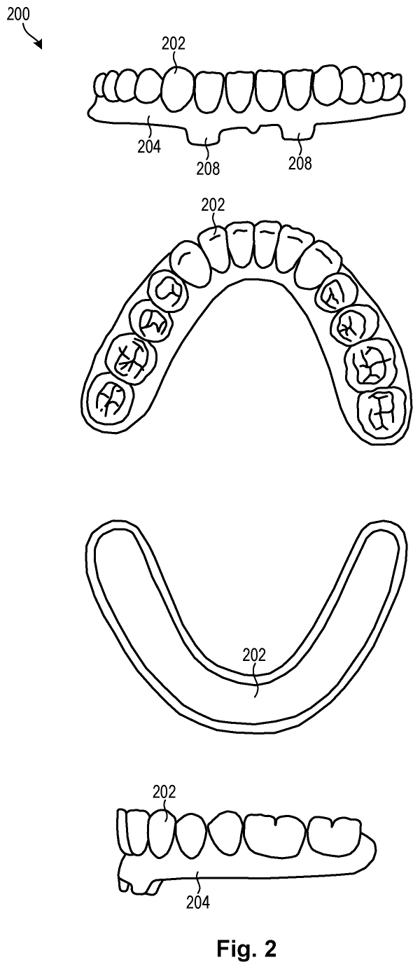

FIG. 2 shows various views of a lower arch 200 of a full-arch shell temporary in accordance with the invention, and more particularly, a front view, top view, bottom view, and side view of the lower arch 200. Like the upper arch 100, the lower arch 200 may include a first portion 202 that is made to resemble and function as a person's teeth and a second portion 204 that is made to resemble and function as a person's gums. As further shown in FIG. 2, in certain embodiments, a bottom side of the lower arch 200 may be hollowed out to enable the upper arch 100 to fit over any protruding bone/gums, implant abutments, and the like, when placing the lower arch 200 in the patient's mouth.

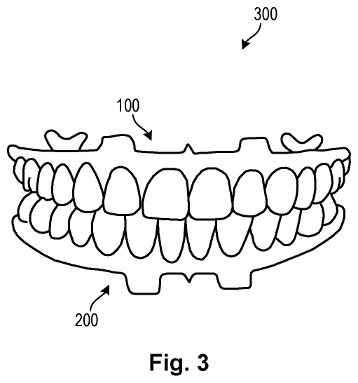

Once the location of the upper arch 100 of the full-arch shell temporary 300 is established, the lower arch 200 of the full-arch shell temporary 300 may be positioned in the patient's mouth relative to the upper arch 100. For example, the lower arch may be placed in occlusion with the upper arch 100 (as shown in FIG. 3) and the patient's lower jaw may be raised until it comes into contact with the lower arch 200. In certain embodiments, the patient's lower jaw may be raised until a mucogingival junction of the lower jaw lines up with reference tabs 208 on the lower arch 200. At this point, the lower arch 200 may be positioned correctly with respect to the patient's lower jaw.

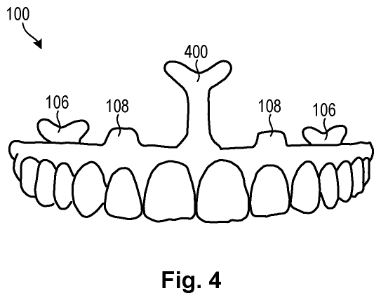

Referring to FIG. 4, in certain embodiments in accordance with the invention, another temporary saddle 400 (also referred to herein as the “nasal spine saddle 400”) may be incorporated into the upper arch 100 to assist in positioning the upper arch 100 in the patient's mouth at an angle that is aesthetically pleasing and anatomically correct. Like the hamular notches, the nasal spine may provide another anatomical landmark in the patient's mouth that may provide a reference point for positioning the upper arch 100.

In certain embodiments, when positioning the upper arch 100 in the patient's mouth, the nasal spine saddle 400 may be configured to rest against the nasal spine under the patient's upper lip to establish the angle of the upper arch 100 relative the hamular notches. The temporary nasal spine saddle 400 along with the temporary hamular notch saddles 106 may together form a stable tripod that firmly establishes a position of the upper arch 100 in the patient's mouth. In certain embodiments, the nasal spine saddle 400 may also have a “Y” shape with the top of the “Y” configured to engage the nasal spine. In certain embodiments, the reference tabs 108 may be used to verify that the upper arch 100 is properly positioned by verifying that they are in line with the mucogingival junction of the upper jaw. In other embodiments, the reference tabs 108 may be removed since the nasal spine saddle 400 may be sufficient to provide the proper angle.

Once a desired position is determined for the upper arch 100 in the patient's mouth, the nasal spine saddle 400, like the hamular notch saddles 106, may be removed from the upper arch 100. This may be accomplished by snapping, cutting, or grinding the nasal spine saddle 400 off of the upper arch 100. In certain embodiments, grooves 500, 502 may be placed at a base of the nasal spine saddle 400 and hamular notch saddles 106 to facilitate breaking off the saddles 106, 400 of the upper arch 100 when they are no longer needed. The upper arch 100 may then be cleaned up and ground or polished where the saddles 106, 400 were broken off.

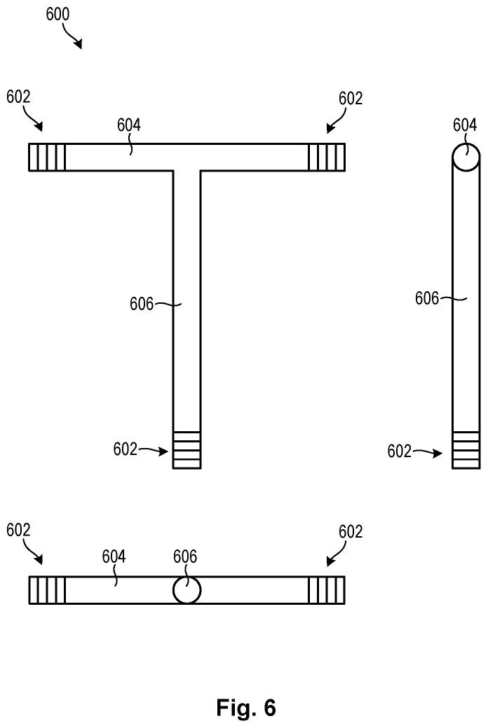

Referring to FIG. 6, in certain embodiments, the full-arch shell temporary 300 may be provided in different sizes to accommodate different sized mouths. For example, the full-arch shell temporary 300 may be provided in small, medium, and large sizes. In practice, the distance between the hamular notches of patients typically varies between about 42 and 46 millimeters. Thus, in one embodiment, the small full-arch shell temporary 300 may measure about 42 millimeters between the hamular notch saddles 106, the medium full-arch shell temporary 300 may measure about 44 millimeters between the hamular notch saddles 106, and the large full-arch shell temporary 300 may measure about 46 millimeters between the hamular notch saddles 106 to accommodate mouths of different sizes.

In certain embodiments, in order to determine what size of full-arch shell temporary 300 a patient may need, a measuring tool 600 may be utilized. FIG. 6 shows one example of such a measuring tool 600, also referred to herein a “hamulometer 600.” A front view, side view and bottom view of the hamulometer 600 are shown. As shown, the hamulometer 600 may have a “T” shape and may include various markings 602 on the ends of the “T” shape. These marking 602 may assist a dentist in measuring the size of a patient's mouth in order to determine what size of full-arch shell temporary 300 the patient may need. A top portion 604 of the hamulometer 600 and its markings 602 may be used to measure a distance between the hamular notches of the patient. By contrast, a vertical portion 606 of the hamulometer 600 and its markings 602 may be used to measure the distance to a front of the patient's jaw or teeth. Based on these measurements, the dentist may determine whether the patient needs the small, medium, or large full-arch shell temporary 300.

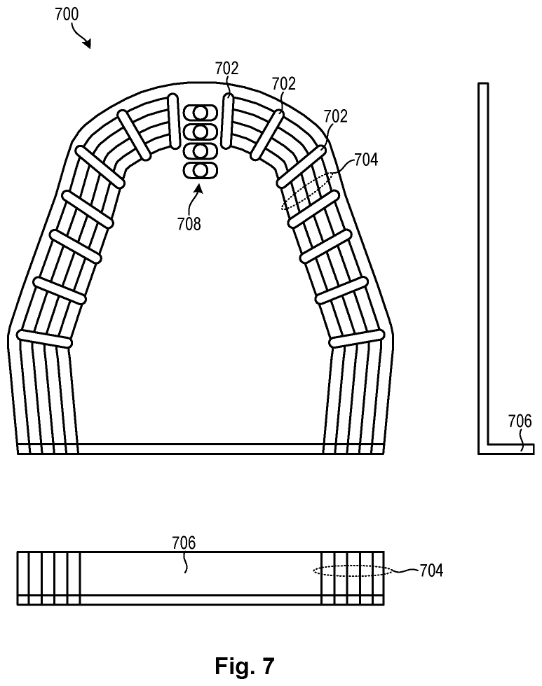

Referring to FIG. 7, once the size of the full-arch shell temporary 300 is determined, a jig-like device may be used to determine where implants should be placed in the jaw of the patient, and more specifically where a dentist wants screw channels to come out for attaching the upper arch 100 of the full-arch shell temporary 300 to the patient's upper jaw. FIG. 7 shows one embodiment of such a jig device 700. A bottom view, side view and rear view of the jig device 700 are shown. As shown, the jig device 700 may include a series of elongated apertures 702 as well as markings 704. A lip 706 of the jig device 700 may be placed in the hamular notches of the patient with the markings lined up with the hamular notches depending on whether the patient has small, large, or medium spacing between his or her hamular notches.

In certain embodiments, the jig device 700 is fabricated from a substantially clear material (with the exception of the markings 704) so that the dentist can view the hamular notches as well as the rest of the jaw through the jig device 700. Any of the apertures 702 may provide candidates for implant and/or screw channel placement in the patient's mouth. Where the implant and/or screw channels are placed within the apertures 702 may depend on the location of the corresponding markings 704 and the size of the patient's mouth that was determined with the hamulometer 600. For example, the inner markings may be used for a patient with a small mouth and the outer markings may be used for a patient with a larger mouth. A marking tool may be used to mark locations of the implants/screw channels through the apertures 702. In certain embodiments, various other apertures 708 may be configured to align with the incisive papilla of the patient to verify that the jig device 700 is properly placed and aligned in the patient's mouth.

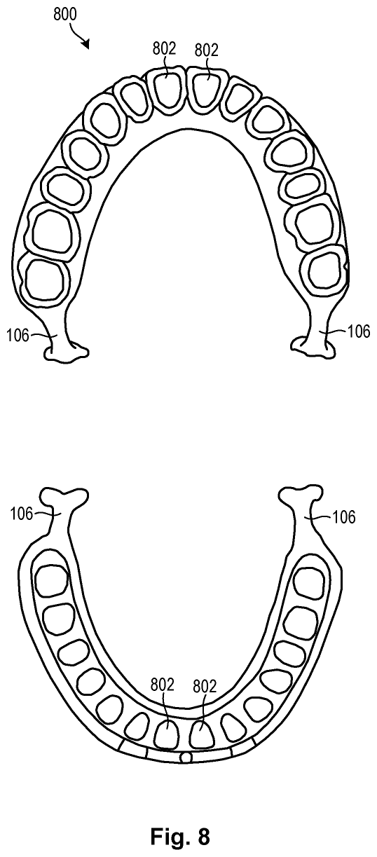

Referring to FIG. 8, an alternative to the jig device 700 of FIG. 7 is shown. In this embodiment, a guide (hereinafter referred to as a “shell guide”) may be used to determine where implants should be placed in the jaw of the patient, and more specifically where screw channels are placed to attach the upper arch 100 to the patient's upper jaw. FIG. 8 shows one embodiment of the upper arch 800 of the shell guide. A bottom view and top view are shown. As shown, the upper arch 800 of the shell guide roughly resembles the upper arch 100 of the full-arch shell temporary 300 illustrated in FIG. 1, except that holes 802 are formed in the locations of the teeth. These holes 802 may provide candidate locations for implants as well as screw channels in the upper arch 100 of the full-arch shell temporary 300. In certain embodiments, the shell guide is made of a substantially transparent material to enable the gums and jaw of the patient to be viewed through the shell guide.

In general, prior to fitting a patient with the upper arch 100 of the full-arch shell temporary 300, the upper arch 800 of the shell guide of the correct size (e.g., small, medium, or large) may be placed in the patient's mouth in the same way as the upper arch 100 of the full-arch shell temporary 300, namely by placing the temporary saddles 106 in the hamular notches of the patient. In certain embodiments, a proper angle of the upper arch 800 of the shell guide may be established by aligning reference tabs (not shown) with a mucogingival junction of the patient or by placing a nasal spine saddle (not shown) in contact with a nasal spine of the patient. Once the upper arch 800 of the shell guide is property positioned, locations for the implants and screw channels may be selected through the holes 802, such as by marking desired locations through holes 802. The upper arch 800 of the shell guide may then be removed from the patient's mouth and the implants may be placed at the locations that were marked.

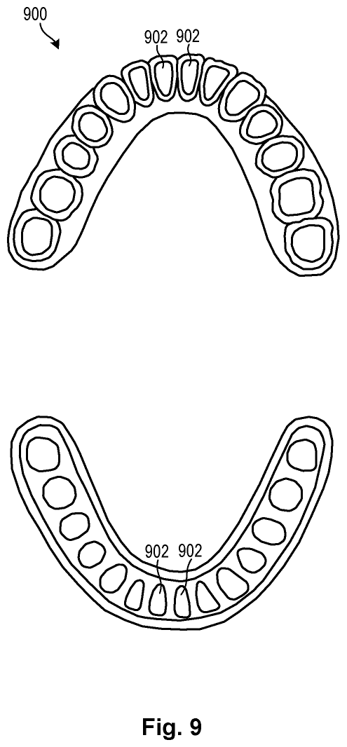

FIG. 9 shows one embodiment of a lower arch 900 of the shell guide. A top view and bottom view are shown. As shown, the lower arch 900 of the shell guide roughly resembles the lower arch 200 of the full-arch shell temporary 300 illustrated in FIG. 2, except that holes 902 are formed in the locations of the teeth. These holes 902 may provide candidate locations for implants as well as screw channels in the lower arch 200 of the full-arch shell temporary 300.

Once the upper arch 100 of the full-arch temporary 300 is fixed in the patient's mouth, the lower arch 900 may be positioned in the patient's mouth relative to the upper arch 100. More specifically, the lower arch 900 of the shell guide may be placed in occlusion with the upper arch 100 and the patient's lower jaw may be raised until it comes into contact with the lower arch 900. In certain embodiments, the patient's lower jaw may be raised until a mucogingival junction of the lower jaw lines up with reference tabs on the lower arch 900. At this point, the lower arch 900 may be positioned correctly with respect to the patient's lower jaw and the holes 902 in the lower arch 900 may be used to determine locations for implants in the lower jaw and screw channels in the lower arch 200 of the full-arch shell temporary 300.

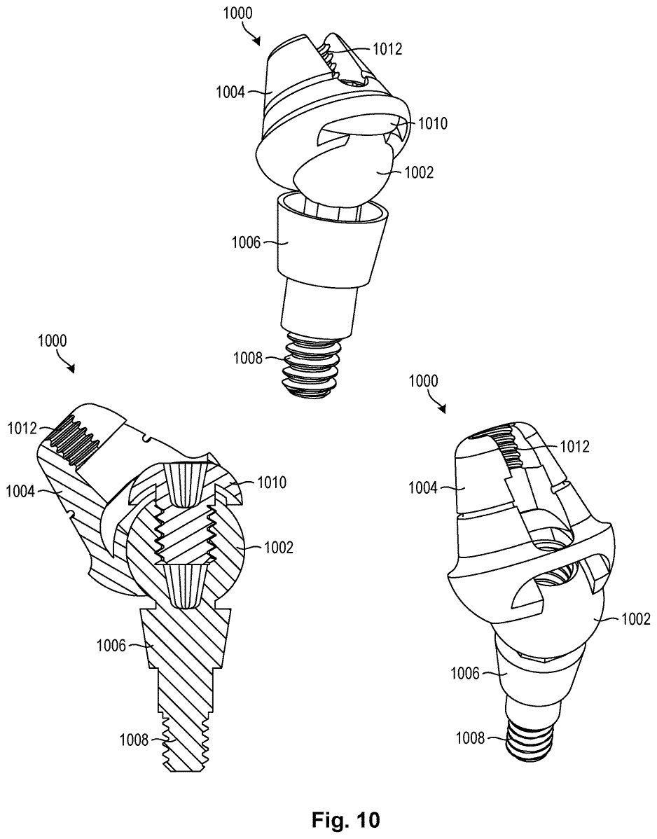

Referring to FIG. 10, once the locations of implants are determined in the patient's mouth, holes may be drilled in the patient's jaw bone and implants may be placed in the holes. These implants may provide anchors for the full-arch shell temporary 300. In certain embodiments, four implants may be placed in both the upper and lower jaw to provide anchors for the full-arch shell temporary 300.

Once the implants are placed in the jaw of a patient, multi-unit abutments may be used to correct for height variations in the implants, and to establish a level platform for the full-arch shell temporary 300 as well as the eventual permanent prosthetic teeth. The multi-unit abutments may also be used to provide a connection to the implants that is even with or just below the gingival surface. In certain cases, angled multi-unit abutments may be used to correct the angle of implants while also keeping screw channels away from facial surfaces of the full-arch shell temporary 300. Nevertheless, conventional angled multi-unit abutments are typically only available in certain fixed angles, such as 17 and 30 degree angles. Unfortunately, these fixed angles may not be ideal in all cases, such as where other intermediate angles would provide a more optimal result.

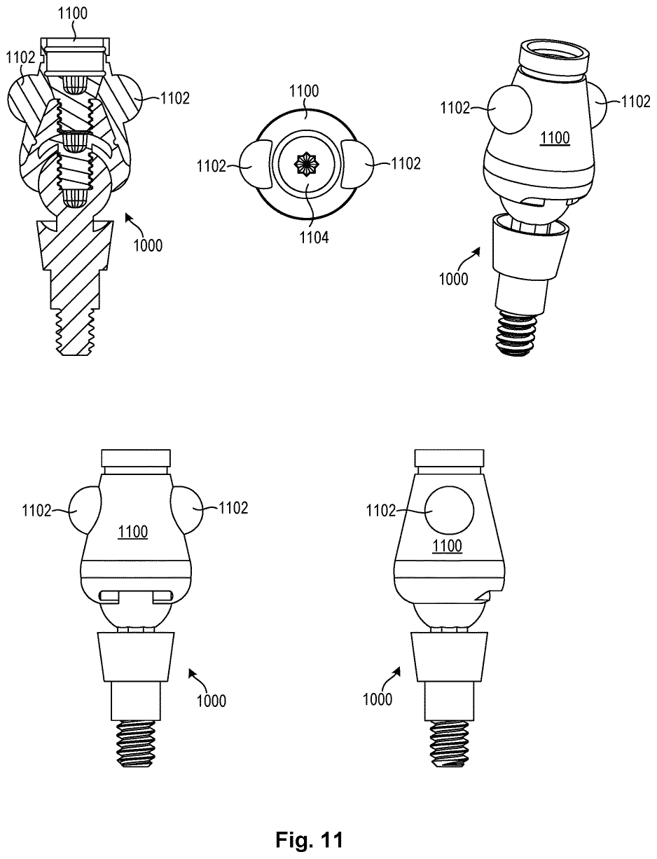

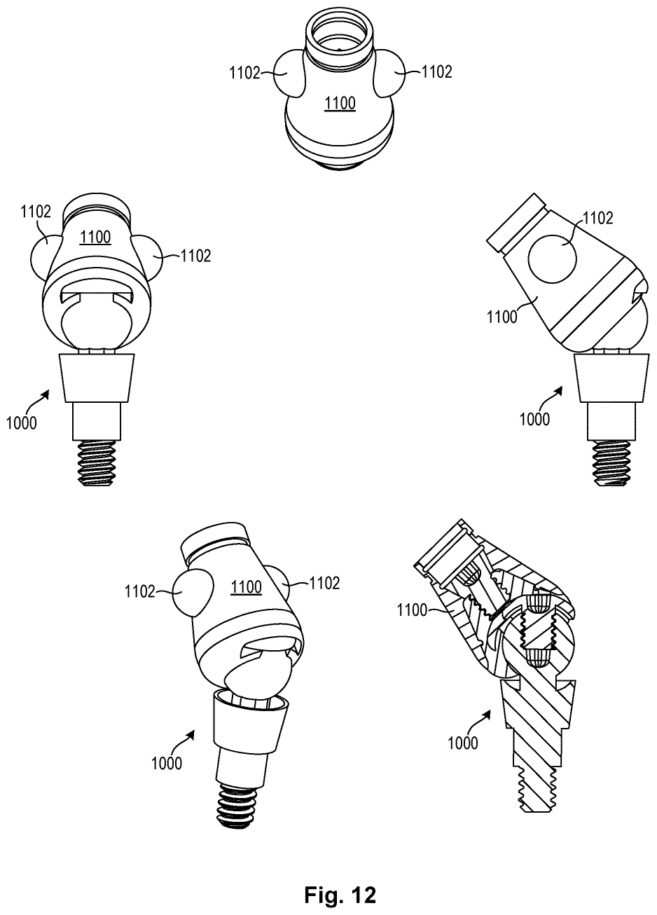

In certain embodiments in accordance with the invention, an adjustable-angle multi-unit abutment may be provided to provide a much greater range of angles than may be achieved with a fixed-angle multi-unit abutment. FIG. 10 shows various views of one embodiment of an adjustable-angle multi-unit abutment 1000 in accordance with the invention. As shown, the adjustable-angle multi-unit abutment 1000 may include a ball portion 1002 and a pivoting portion 1004 configured to pivot on the ball portion 1002 to achieve a desired angle. A neck 1006 may extend from the ball portion 1002 and include threads 1008 for screwing into an implant. The pivoting portion 1004 may be internally threaded to receive a cylinder or other structure, as will be discussed in association with FIGS. 11 and 12.

When a desired angle is achieved, a set screw 1010 may be tightened to lock the pivoting portion 1004 relative to the ball portion 1002. In certain embodiments, the set screw 1010 may include a rounded head that is shaped so as not to interfere with the angular adjustment of the pivoting portion 1004. In certain embodiments, a bottom side of the head is shaped to conform to the ball portion 1002.

In certain embodiments, the pivoting portion 1004 of the adjustable-angle multi-unit abutment 1000 may achieve an angle between about 0 and 45 degrees relative to an axis of the neck 1006 and ball portion 1002, thereby allowing a very large (i.e., potentially infinite) number of angles to be achieved. Pivoting the pivoting portion 1004 combined with rotating the ball portion 1002 may be used together to achieve a wide variety of orientations of the adjustable-angle multi-unit abutment 1000. The adjustable-angle multi-unit abutment 1000 may be made from any suitable material, such as various metals, alloys, ceramics, or the like, to achieve a desired strength and rigidity. The adjustable-angle multi-unit abutment 1000 disclosed herein is not limited to use with a full-arch shell temporary 300 but may also be used or modified for use with crowns, bridges, or the like.

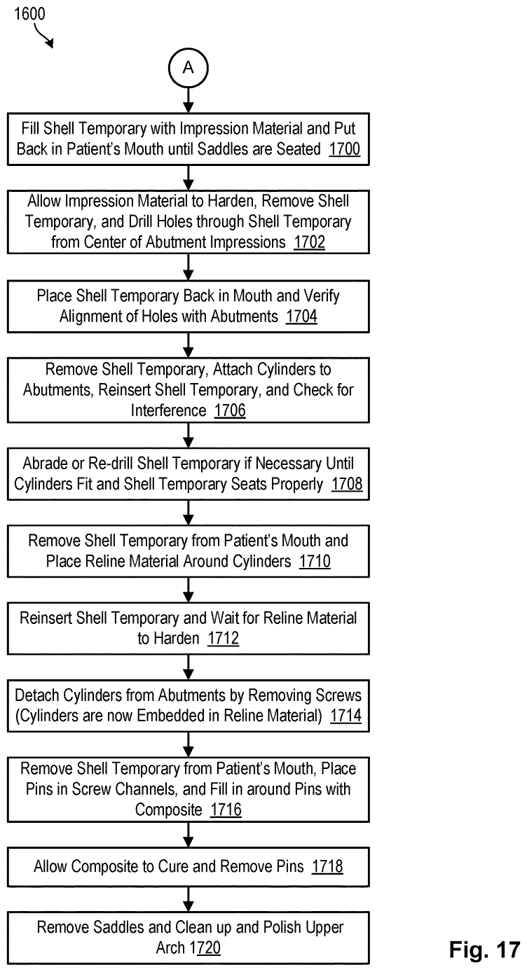

FIGS. 11 and 12 show a cylinder 1100 that is coupled to an adjustable-angle multi-unit abutment 1000. FIG. 11 shows various views of the cylinder 1100 coupled to an adjustable-angle multi-unit abutment 1000 that is locked in a straight position (i.e., a 0 degree angle). FIG. 12 shows various views of the cylinder 1100 coupled to an adjustable-angle multi-unit abutment 1000 that is locked in an angled position (i.e., between about 0 and 45 degrees). As will be explained in more detail hereafter in association with FIGS. 16 and 17, the cylinder 1100 may be embedded within a full-arch shell temporary 300 with reline material or another composite to enable the full-arch shell temporary 300 to be attached to the adjustable-angle multi-unit abutment 1000. In certain embodiments, features 1102, such as the half-sphere shapes 1102, may be incorporated into the cylinder 1100 so that the cylinder 1100 is firmly embedded and gripped within the reline material of the full-arch shell temporary 300. Once embedded in the full-arch shell temporary 300, a screw 1104 or other fastener 1104 may be used to attach the cylinder 1100 to the adjustable-angle multi-unit abutment 1000.

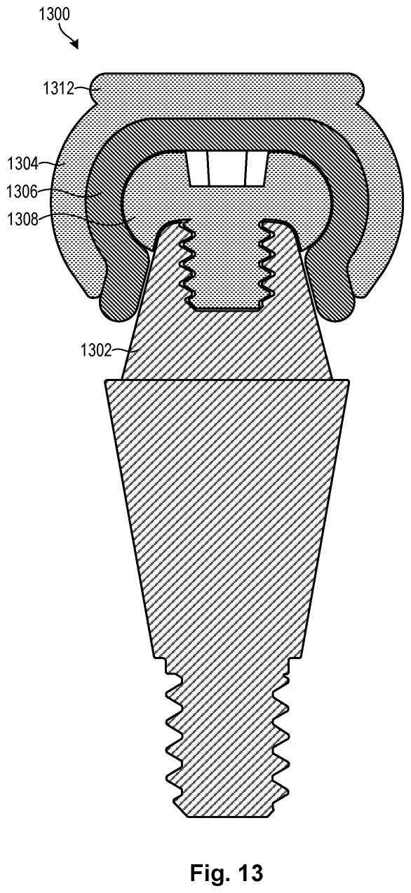

Referring to FIG. 13, in certain embodiments, it may be desirable to have a full-arch shell temporary 300, and more specifically an upper arch 100 and lower arch 200 of the full-arch shell temporary 300, that can be quickly fixed and/or removed from the patient's mouth. In certain embodiments, it may be desirable to have a full-arch shell temporary that will simply snap into place in the patient's mouth, or quickly unsnap to remove the full-arch shell temporary from the patient's mouth.

In order to provide the aforementioned characteristics, in certain embodiments, a snap cap 1300 may be provided in lieu of the cylinder 1100 described in association with FIGS. 11 and 12. This snap cap 1300, like the cylinder 1100, may be embedded with the reline material of the full-arch shell temporary 300, and may connect to an abutment or other structure screwed into an implant. FIG. 13 shows one contemplated embodiment of a snap cap 1300 that may be used to couple a full-arch shell temporary to an abutment 1302. As shown, the snap cap 1300 may, in certain embodiments, include a cap portion 1304 and a pliable insert 1306. The cap portion 1304 may be substantially more rigid than the pliable insert 1306.

In this particular embodiment, the pliable insert 1306 fits over a rounded screw 1308 or other rounded structure that screws into the abutment 1302. Thus, the snap cap 1300 may fit over a rounded structure 1308. In certain embodiments, the pliable insert 1306 is made from a material such as silicone that replicates the bounce, elasticity, and shock absorption of real teeth provided by the periodontal ligament. In other words, natural teeth are not directly connected to bone and the pliable insert 1306 is intended to mimic the periodontal ligament that provides the bounce, elasticity, and shock absorption of natural teeth relative to the jaw bone.

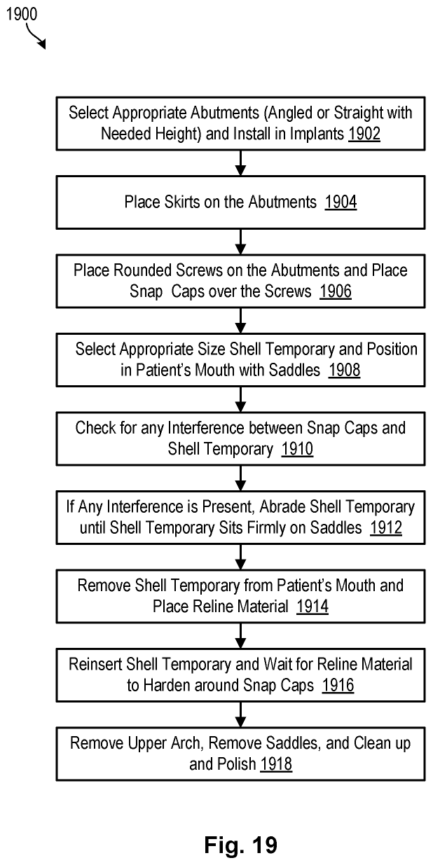

As will be explained in more detail as it relates to FIG. 19, in order to enable a full-arch shell temporary to easily snap on or off of the abutments, the cap portion 1304 may be embedded in the full-arch shell temporary 300. This may be accomplished using reline material that is used to fill the gap or space between the snap caps 1300 and the upper arch 100 or lower arch 200 of the full-arch shell temporary 300. In certain embodiments, the cap portion 1304 may include some feature 1312 (in this embodiment a ridge 1312) that enables the snap cap 1300 to make a good connection with the reline material and thus a good connection with the full-arch shell temporary 300.

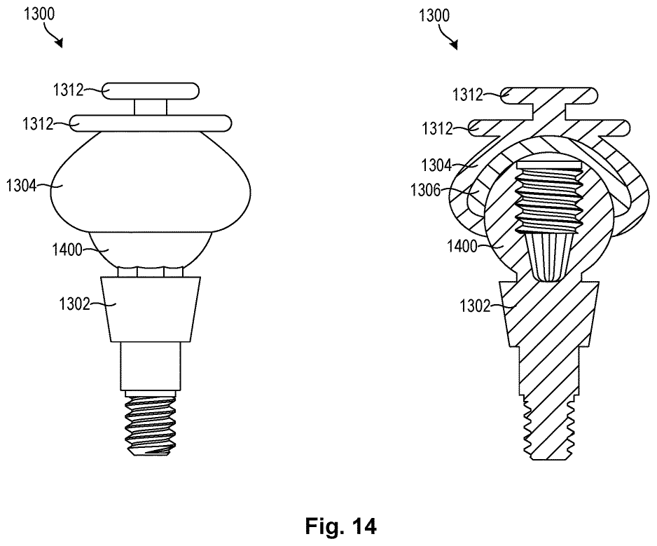

Referring to FIG. 14, an alternative embodiment of the snap cap 1300 is illustrated. In this embodiment, the abutment 1302 may have a rounded top 1400 to accommodate the snap cap 1300. Like the previous example, the snap cap 1300 may include a cap portion 1304 and a pliable insert 1306, such as a silicone insert 1306. In this particular embodiment, the pliable insert 1306 fits over the rounded top 1400. In this embodiment, the cap portion 1304 includes ridge-like features 1312 that enable the snap cap 1300 to be firmly embedded within the reline material and thus create a good connection with the full-arch shell temporary 300. Compared to the embodiment of FIG. 13, the embodiment illustrated in FIG. 14 may be simpler and provide a lower profile since it eliminates the rounded screw 1302 and incorporates the rounded surface 1400 or ball 1400 directly into the abutment 1302.

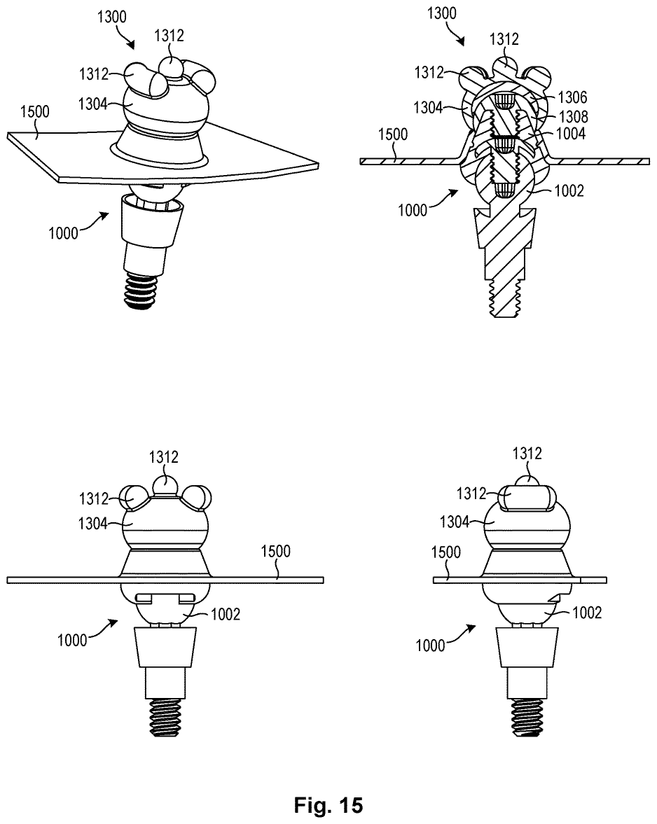

Referring to FIG. 15, another alternative embodiment of the snap cap 1300 is illustrated. This embodiment is similar to that illustrated in FIG. 13. Thus, the snap cap 1300 includes a cap portion 1304 and a pliable insert 1306, with the cap portion 1304 being substantially more rigid than the pliable insert 1306. The pliable insert 1306 fits over a rounded screw 1308 that conforms to an outer contour of the abutment 1000. In this embodiment, the abutment 1000 is an adjustable-angle multi-unit abutment 1000 as previously described in association with FIG. 10. In the illustrated embodiment, the cap portion 1304 includes various features 1312 that enable the snap cap 1300 to be embedded in reline material and thus create a good connection with the full-arch shell temporary.

One notable addition to this embodiment of the snap cap 1300 and abutment 1000 is the skirt 1500. When placing the reline material between the full-arch shell temporary 300 and the cap portion 1304 of the snap cap 1300, the skirt 1500 may keep the reline material from coming into contact with the abutment 1000 and potentially interfering with the connection between the snap cap 1300 and the abutment 1000, or interfering with the ability of the pivoting portion 1004 to pivot on the ball portion 1002. In general, the skirt 1500 may keep the reline material confined to areas where it is needed and prevent it from coming into contact with items or objects where it is not needed or wanted. In addition, the skirt 1500 may keep undercuts, blood, saliva, stitches etc. in the patient's mouth from coming into contact with the reline material. Once the reline material has cured, the snap cap 1300 may be removed from the abutment 100 and the skirt 1500 may be removed since it is no longer needed.

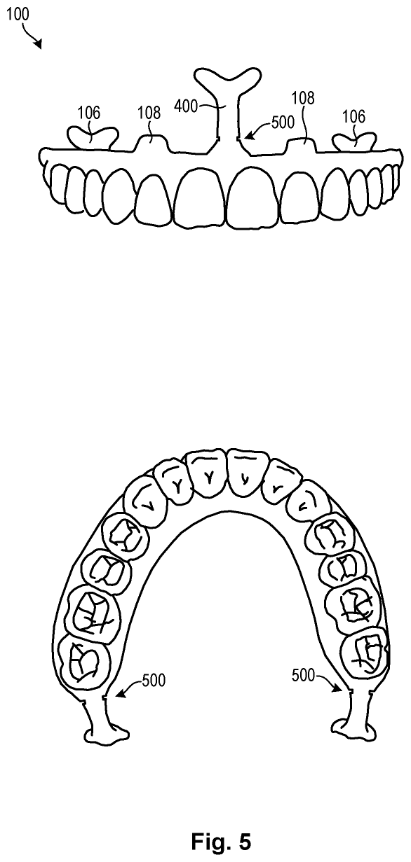

Referring to FIGS. 16 and 17, one embodiment of a method 1600 for fitting a patient with an upper arch 100 of a full-arch shell temporary 300 is illustrated. This method 1600 may utilize many of the structures disclosed in FIGS. 1 through 15. As shown, using the method 1600, a dentist may initially use 1602 the hamulometer 600 or another measuring tool to determine a size of shell guide and full-arch shell temporary 300 to utilize with the patient. For example, the dentist may determine if the dimensions of the patient's mouth are small, medium, or large as previously discussed. If needed, the dentist may remove 1604 any remaining teeth of the patient. That dentist may then select 1606 an appropriately sized shell guide in order to determine placement of implants in the patient's mouth. An upper arch 800 of the shell guide may then be positioned in the patient's mouth using the two hamular notch saddles 106 and the nasal spine saddle 400. Specifically, the dentist may place the two hamular notch saddles 106 against the patient's hamular notches and the nasal spine saddle 400 against the patient's nasal spine in order to seat the upper arch 800 of the shell guide in the patient's mouth.

At this point, the dentist may check 1610 for any interference between the shell guide and the patient's mouth. That is, the dentist may check whether the upper arch 800 of the shell guide is able to be seated properly on the patient's hamular notches and nasal spine or if the anatomy of the patient's mouth prevents that from occurring. If interference is present, the dentist may abrade 1610 (e.g., grind, cut, remove, etc.) portions of the patient's jawbone, tissue, or the like in the locations where the interference is occurring until the upper arch 800 of the shell guide can be properly seated on the patient's hamular notches and nasal spine. Once the upper arch 800 of the shell guide is seated properly, the dentist may mark 1612 locations for implant placement using the shell guide as a template. This may be done with a marker or a tool to poke the gums and cause slight bleeding, for example.

The dentist may then proceed to place 1614 the implants as known to those of skill in the art of dentistry. In general, this process may involve drilling holes in the upper jawbone of the patient and threading the implants into the holes that have been created. Appropriate abutments (either straight or angled) may then be selected 1616 and installed 1616 in the implants. In certain embodiments, the adjustable-angle multi-unit abutment 1000 previously described may be selected and installed to provide a more precise angle where needed.

The dentist may then select 1618 an appropriately sized full-arch shell temporary 300 (e.g., small, medium, or large) and position 1618 the upper arch 100 of the full-arch shell temporary 300 in the patient's mouth using the hamular notch saddles 106 and nasal spine saddle 400. The dentist may then check 1620 for any interference between the upper arch 100 and the abutments that were previously installed to determine if the abutments are preventing the upper arch 100 from properly seating on the hamular notches and nasal spine of the patient. If any interference is present, the dentist may abrade 1622 (e.g., grind down, cut, etc.) any portions of the upper arch 100 that are interfering with the abutments until the interference is eliminated or reduced to an acceptable amount.

At this point, the dentist may fill 1700 the upper arch 100 of the full-arch shell temporary 300 with impression material (e.g., Blu-Mousse® impression material) and place 1700 the upper arch 100 back in the patient's mouth until the hamular notch saddles 106 and nasal spine saddle 400 are seated. This will allow the installed abutments to make indentations in the impression material with the upper arch 100 in its intended position. Once the impression material has hardened 1702, the upper arch 100 may be removed 1702 from the patient's mouth. The dentist may then drill 1702 holes through the upper arch 100 in the locations of the indentations. The upper arch 100 may then be placed 1704 back in the patient's mouth and the dentist may verify 1704 that the newly drilled holes align with the abutments. If so, the dentist may remove 1706 the upper arch 100 from the patient's mouth, attach 1706 cylinders 1100 to the abutments (e.g., with screws), and then reinsert 1706 the upper arch 100 into the patient's mouth to check for any interference. If any interference is present, the dentist may abrade 1708 or redrill 1708 the upper arch 100 until the cylinders 1100 fit and the upper arch 100 is properly seated.

At this point, the dentist may remove 1710 the upper arch 100 from the patient's mouth and place reline material around the cylinders 1100. Alternatively, or additionally, the reline material may be placed in the hollowed out portion of the upper arch 100. The upper arch 100 may then be reinserted 1712 into the patient's mouth and seated while the reline material hardens around the cylinders 1100. The dentist may then detach 1714 the cylinders 1100 from the abutments by removing the screws that couple the two together. At this point, the cylinders 1100 will be embedded in the reline material and thus securely coupled to the upper arch 100 of the full-arch shell temporary 300.

The dentist may then remove 1716 the upper arch 100 from the patient's mouth and place 1716 pins in the screw channels. In certain embodiments, an outer diameter of the pins is slightly larger than the outer diameter of the screws that are used to attach the cylinders 1100 to the abutments. Composite may then be filled in 1716 around the pins and allowed 1718 to cure. The pins may then be removed 1718 from the upper arch 100. This will leave nicely formed screw channels with a diameter that is just larger than that needed to accommodate the screws used to attach the cylinders 1100 to the abutments. At this point, the dentist may remove 1720 the hamular notch saddles 106 and the nasal spine saddle 400 from the upper arch 100 by breaking or cutting them off. The upper arch 100 may then be cleaned up 1720 and polished 1720. The upper arch 100 is then ready to be fixed in the patient's mouth for use as a temporary prosthesis until the patient can be fitted with a final prosthesis.

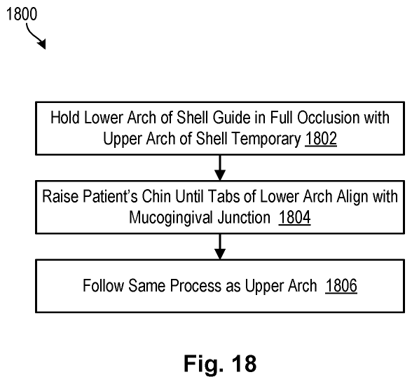

Referring to FIG. 18, a process flow diagram showing one embodiment of a method 1800 for fitting a patient with a lower arch 200 of a full-arch shell temporary 300 is illustrated. As shown, once the patient is fitted with the upper arch 100 of the full-arch shell temporary 300 and the upper arch 100 is installed, the dentist may hold 1802 the lower arch 900 of the shell guide in the patient's mouth in occlusion with the upper arch 100. The dentist may then raise 1804 the patient's chin until the tabs of the lower arch 900 align with the patient's mucogingival junction. The dentist may then proceed 1806 in the same manner as was performed for the upper arch of the full-arch shell temporary 300 as described in FIGS. 16 and 17 except that instead of using the hamular notches and nasal spine as guides, the lower arch is referenced from the placement of the upper arch 100 of the full-arch shell temporary 300. For example, when placing the impression material on the lower arch 200 of the full-arch shell temporary 300, the lower arch 200 may be placed in occlusion with the upper arch 100 to ensure that the impressions and the resulting screw channels are correctly positioned. This will ensure that the lower arch 200 when fixed will form a proper bite with the upper arch 100. This same technique may also be used when placing the reline material on the lower arch 200.

Referring to FIG. 19, in certain embodiments, the method 1600 illustrated in FIGS. 16 and 17 may be modified to work with the snap caps 1300 previously described. That is, instead of drilling holes through the upper and lower arches 100, 200 and embedding cylinders 1100 in the upper and lower arches 100, 200 for attachment to the abutments, snap caps 1300 may alternatively be embedded within the upper and lower arches 100, 200 to enable the upper and lower arches 100, 200 to snap on and off the abutments. FIG. 19 shows one embodiment of a method 1900 for integrating snap caps 1300 into the upper and lower arches 100, 200.

As shown, after the implants have been placed in the patient's jaw, the dentist may select appropriate abutments (such as any of the abutments illustrated in FIG. 13-15) for use with the snap caps 1300. These may be straight or angled and with a desired height. The abutments may then be installed 1902 in (e.g., threaded into) the implants. Skirts 1500 may then be placed 1904 on the abutments. At this point, rounded screws may be placed 1906 on (e.g., threaded into) the abutments unless the top of the abutments are already rounded or ball-shaped like those shown in FIG. 14. The snap caps 1300 may then be placed 1906 over the rounded screws or round tops of the abutments.

At this point, the dentist may select an appropriately-sized full-arch shell temporary 300 and position the upper arch 100 of the full-arch shell temporary 300 in the patient's mouth using the hamular notch saddles 106 and nasal spine saddle 400 previously discussed. The dentist may check 1910 for any interference between the snap caps 1300 and the upper arch 100. If any interference is present, the dentist may abrade 1912 (i.e., grind, cut, etc.) the upper arch 100 of the full-arch shell temporary 300 until the upper arch 100 will sit firmly on the hamular notches and nasal spine.

The upper arch 100 of the full-arch shell temporary 300 may then be removed 1914 from the patient's mouth and reline material may be placed 1914 around the snap caps 1300 and/or within the hollow portion of the upper arch 100. The upper arch 100 may then be reinserted 1916 into the patient's mouth and seated using the saddles 106, 400. The reline material may then be allowed to cure. Once cured, the snap caps 1300 will be embedded in the reline material and securely coupled to the upper arch 100. At this point, the upper arch 100 may be removed 1918 from the patient's mouth (i.e., by snapping the snap caps 1300 off of their respective abutments). The saddles 106, 400 may then be removed 1918 from the upper arch 100 and the upper arch 100 may be cleaned up 1918 and polished 1918.

Once the patient is fitted with the upper arch 100 of the full-arch shell temporary 300, a similar process may be used to fit the lower arch 200 except that instead of using the hamular notches and nasal spine as guides, the lower arch 200 may be positioned relative to the placement of the upper arch 100. This may be accomplished by placing the lower arch 200 in occlusion with the upper arch 100 similarly to what was described in association with FIG. 18.

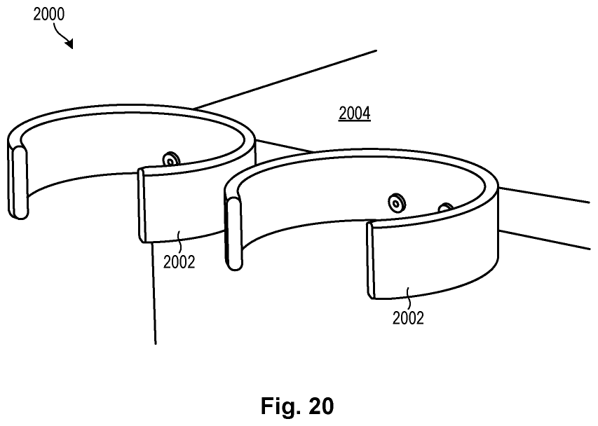

Referring to FIG. 20, when working in a dental office, a dentist is oftentimes moving between patients in different rooms and chairs in order to provide dental care to different patients. In some cases multiple procedures may be going on at the same time. In most cases, the dentist will need to wear gloves, such as latex gloves, since the dentist will often come into contact with blood, saliva, microorganisms, or other potentially harmful substances. In some cases, a dentist that is treating a patient may need to remove his or her gloves to perform a task such accessing reading materials, supplies, or equipment in the dental office. In such cases the dentist may need to temporarily remove gloves only to have to put them on again when he or she returns to the patient. In other cases, a new pair of gloves may be needed each time the dentist moves from one patient to another. This constant need to put on and take off gloves can consume a significant amount of the dentist's time and reduce the dentist's efficiency.

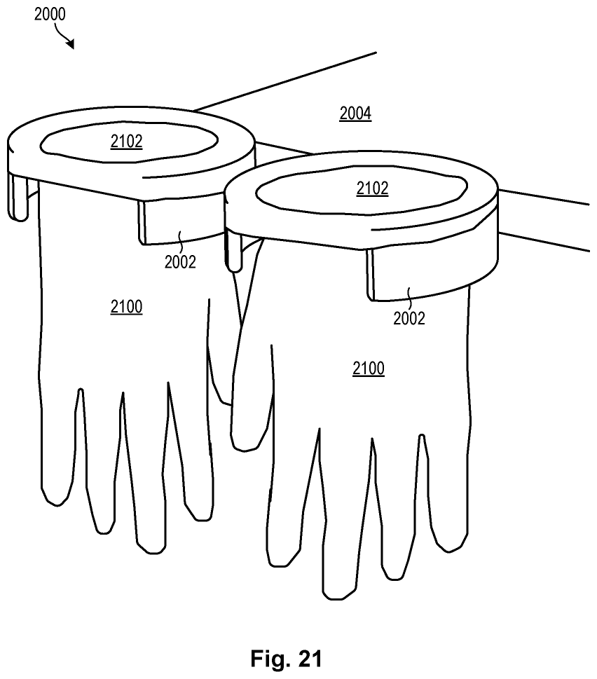

Thus, it would be an advance in the art to provide novel apparatus and methods to enable a dentist or healthcare worker to quickly put on gloves. FIG. 20 is a perspective view showing one embodiment of a glove holder 2000 in accordance with the invention that may be used to facilitate quickly putting on gloves. As shown, in certain embodiments, the glove holder 2000 includes a pair of rings 2002. In this embodiment, the rings 2002 are open to enable a user's hands or arm to pass through the rings 2002. As shown, the rings 2002 may, in certain embodiments, be attached to a countertop 2004 or other structure 2004 with screws or other fasteners. As shown in FIG. 21, the openings of latex or other elastic gloves 2100 may be stretched over the rings 2002 in preparation for use by a dentist or other healthcare worker. In certain embodiments, the gloves 2100 may be positioned on the rings 2002 such that the thumbs are directed toward one another, as shown in FIG. 21. In certain embodiments, an assistant may preplace the gloves 2100 over the rings 2002 in preparation for a dentist or other healthcare worker entering a room.

When the user enters the room where the glove holder 2000 is situated, the user may place his or her hands into the openings 2102 of the gloves 2100 and direct his or her hands downward into the corresponding fingers of the gloves 2100. As the user's fingers reach the bottom of the glove fingers, the gloves 2100 may travel downward which may pop the gloves 2100 off of the rings 2002. This may cause the gloves 2100 to contract around the user's hands. Thus, the user may put on the gloves 2100 in one fast and easy motion without having to hold or handle the gloves 2100 while putting them on. Because the openings 2102 of the gloves 2100 are initially stretched across the rings 2002, the user may be provided a significantly wider opening into which to place his or her hands, thereby speeding up the process of putting on the gloves 2100.

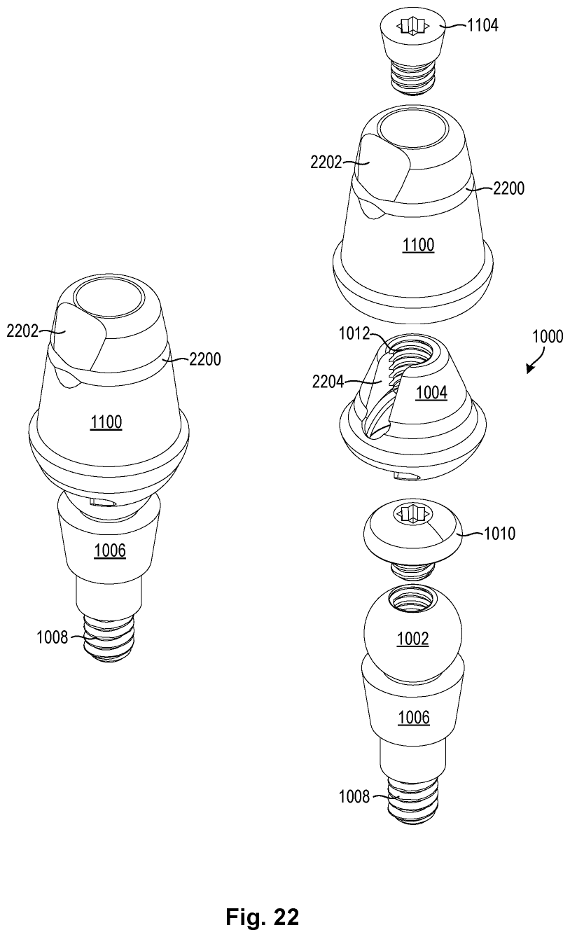

Referring to FIG. 22, another embodiment of a cylinder 1100 for attachment to various types of prosthetic dental devices is illustrated. In certain embodiments, it may be advantageous to provide a cylinder 1100 that is more universal with regard to the types of prosthetic dental devices with which it may be used or interface. As previously discussed, the cylinder 1100 that was disclosed in FIGS. 11 and 12 was designed primarily for embedding within a full-arch shell temporary 300 with reline material or some other composite to enable the full-arch shell temporary 300 to be attached to the adjustable-angle multi-unit abutment 1000. It would be an advance in the art to provide a cylinder 1100 that is not only useful with full-arch shell temporaries 300, but also with other types of prosthetic dental devices such as crowns and bridges. FIG. 22 shows one example of such a cylinder 1100, coupled to an adjustable-angle multi-unit abutment 1000, that may be embedded with prosthetic dental devices such as full-arch shell temporaries 300, crowns, bridges, and the like, with reline material and/or other composites.

As shown, the adjustable-angle multi-unit abutment 1000 includes the pivoting portion 1004 that is configured to pivot on the ball portion 1002 (also referred to herein as a “stationary portion 1002” since it rigidly mounts to a jaw of a patient). The set screw 1010 may be tightened to lock the angle of the pivoting portion 1004 relative to the ball portion 1002. A slot 2204 may be formed in the pivoting portion 1004 to enable a driver to turn the set screw 1010 regardless of the angle of the pivoting portion 1004 relative to the ball portion 1002. As shown, the cylinder 1100 may be attached to the pivoting portion 1004 with a screw or other fastener 1104. In certain embodiments, the cylinder 1100 includes one or more surface features, such as the circumferential groove 2200, to enable the cylinder 1100 to be gripped by the reline material associated with a prosthetic dental device.

In certain embodiments, the cylinder 1100 may also be configured to function as a scan body. That is, the cylinder may be configured for 3-D scanning so that a prosthetic dental device can be properly built to fit over the cylinder 1100 as well as potentially other cylinders 1100 in the patient's mouth. In certain embodiments, a surface feature such as the illustrated planar surface 2202 may be incorporated into the cylinder 1100 so that a scanning device may detect and identify a rotational orientation of the cylinder 1100 when scanning cylinders 1100 within a patient's mouth.

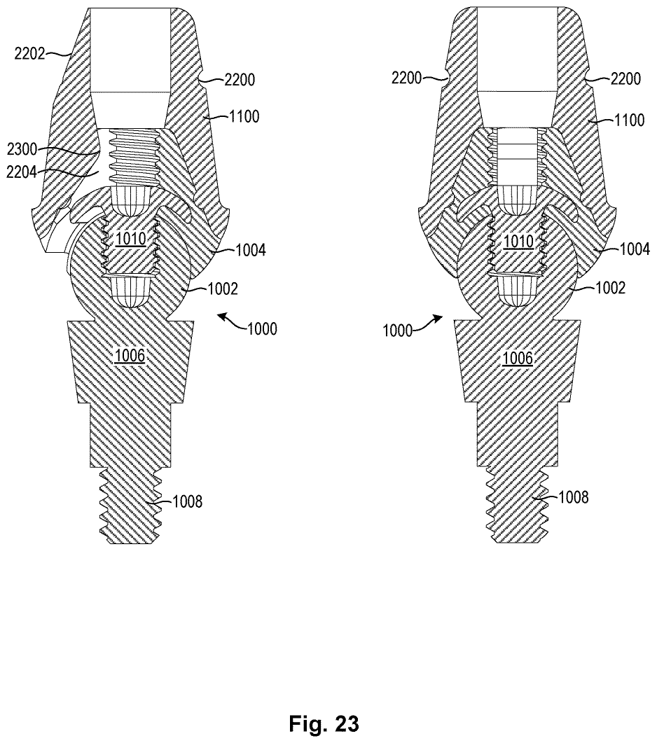

FIG. 23 shows cross-sectional views of the cylinder 1100 and adjustable-angle multi-unit abutment 1000 shown in FIG. 22. As shown in FIG. 23, in certain embodiments, an internal surface of the cylinder 1100 may include a projection 2300 that protrudes into the slot 2204 of the pivoting portion 1004. This may prevent the cylinder 1100 from rotating relative to the pivoting portion 1004 (i.e., rotationally fix the cylinder 1100 relative to the pivoting portion 1004).

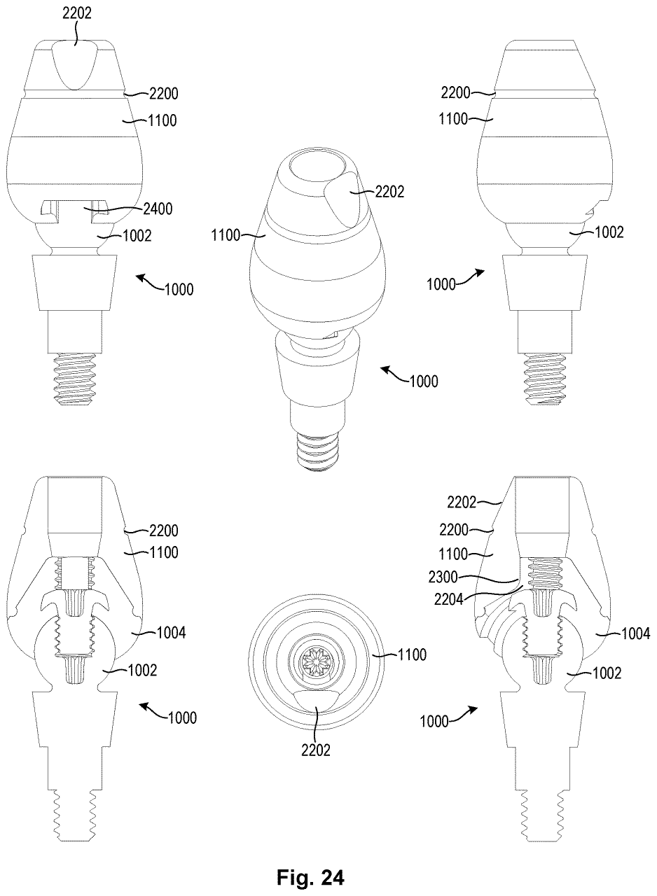

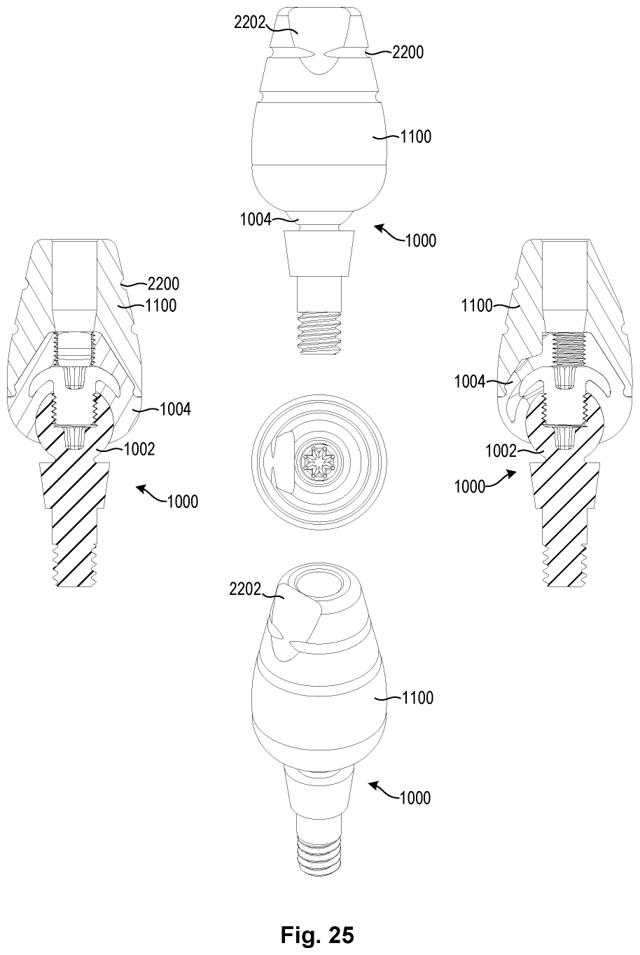

FIG. 24 shows various views of another embodiment of a cylinder 1100 that may be attached to an adjustable-angle multi-unit abutment 1000 in accordance with the invention. As shown, in this embodiment, the cylinder 1100 and pivoting portion 1004 include a slot 2400 or groove 2400 to enable the set screw 1010 to be inserted therethrough so that it can be positioned within the adjustable-angle multi-unit abutment 1000. In other embodiments, such as the embodiment illustrated in FIG. 25, the slot 2400 or groove 2400 may be omitted from the design at least to the extent of it being visible from an outer surface of the pivoting portion 1004 and/or cylinder 1100. This may occur in embodiments where the adjustable-angle multi-unit abutment 1000 is laser welded together after the set screw 1010 is positioned therein. This may eliminate the need for the slot 2400 or groove 2400 and may generally provide a more streamlined designed. This may also reduce cavities or openings in the design where tissue, food, saliva, bacteria, and the like can enter or interfere with the cylinder 1100 and adjustable-angle multi-unit abutment 1000.

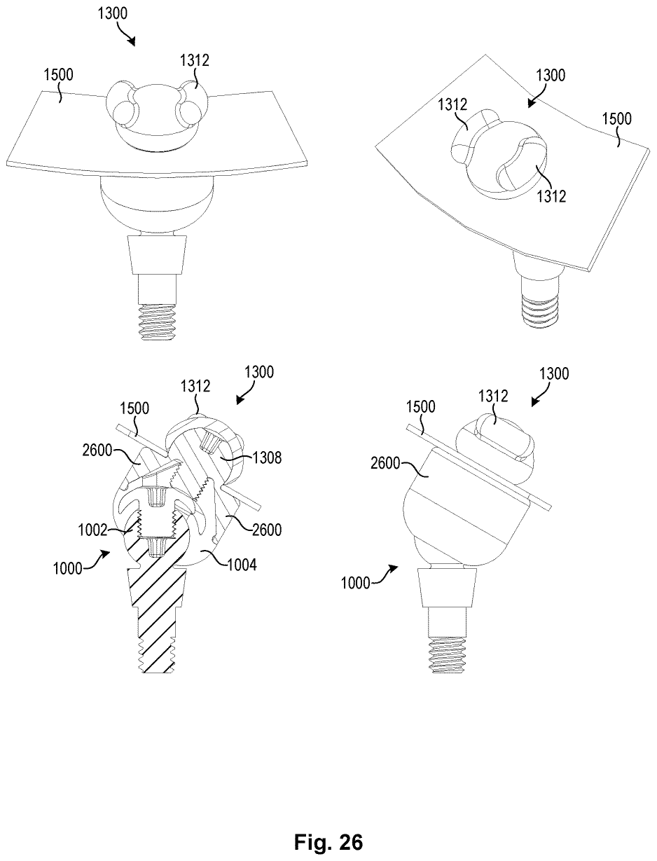

FIG. 26 shows various views of another embodiment of an adjustable-angle multi-unit abutment 1000 incorporating a snap cap 1300, a skirt 1500 that may be used to keep reline material off of the abutment 1000, and a rounded screw 1308 with a skirt 2600 to keep a patient's tissue away from the abutment 1000. As shown in FIG. 26, in certain embodiments, the rounded screw 1308 that was previously described in association with FIG. 15 may be modified to include a skirt 2600 thats extend downward from the rounded screw 1308. An underside of this skirt 2600 may conform to the outer contour of the pivoting portion 1004 of the adjustable-angle multi-unit abutment 1000. This may impart a generally cylindrical shape to the adjustable-angle multi-unit abutment 1000 below the skirt 1500. Among other benefits, this feature may keep tissue away from adjustable-angle multi-unit abutment 1000 while a patient's tissue is healing (i.e., thereby preventing tissue from collapsing over the adjustable-angle multi-unit abutment 1000 during a healing phase). This may provide a desired contour to the patient's tissue as well as keep tissue away from the adjustable-angle multi-unit abutment 1000 so that the adjustable-angle multi-unit abutment 1000 can be used as a scan body within the patient's mouth.

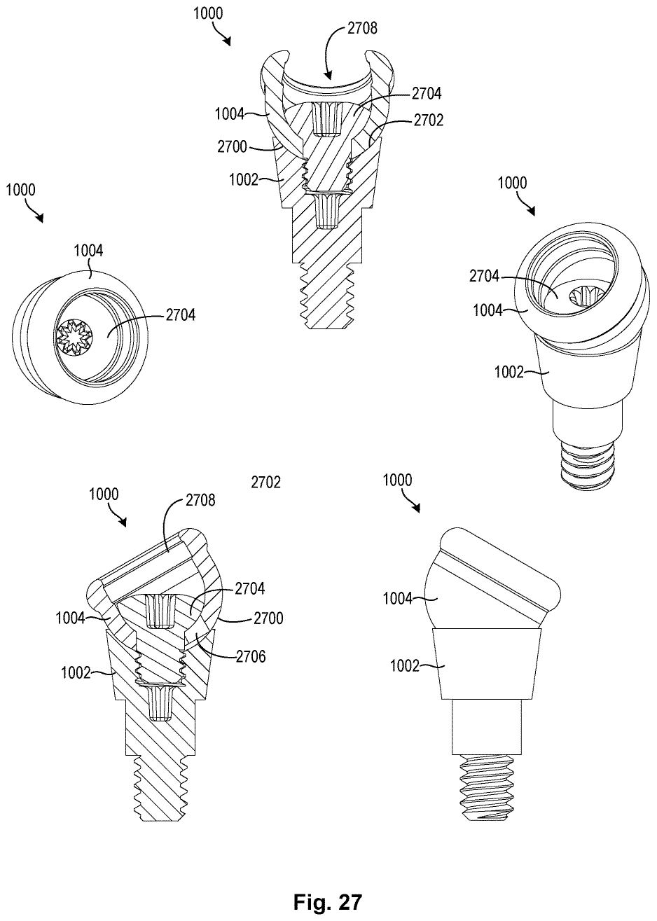

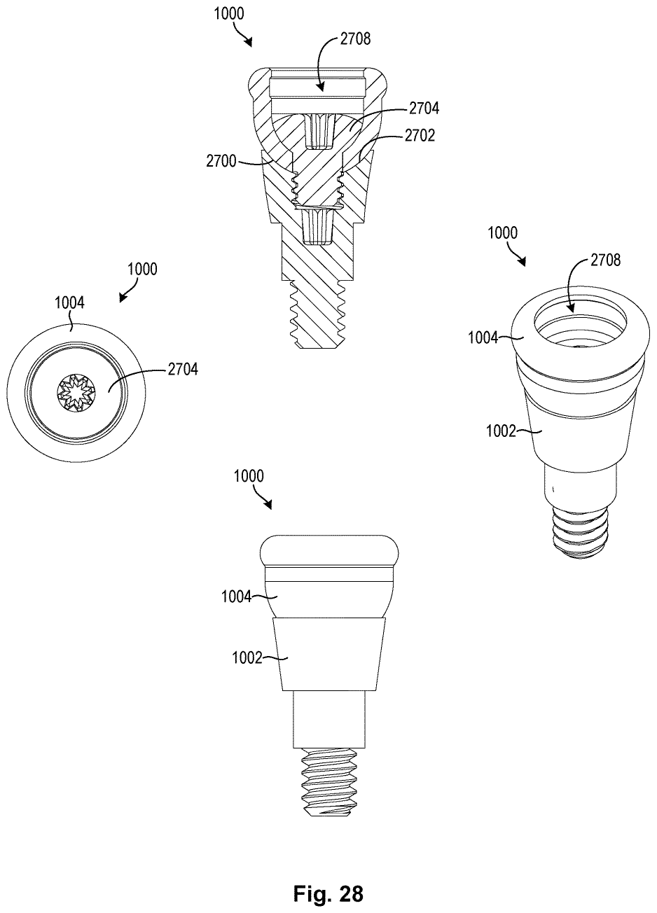

FIG. 27 shows various views of another embodiment of an adjustable-angle multi-unit abutment 1000 in accordance with the invention. In this embodiment, the functionality is inverted such that a ball (or rounded shape) is incorporated into the pivoting portion 1004. This ball 2700 or rounded shape 2700 pivots within a socket 2702 or saddle 2702 of a stationary portion 1002 (i.e., a component that is rigidly mounted to a patient's jaw bone). In certain embodiments, a screw 2704 or other fastener 2704, passing through a slotted hole 2706 in the pivoting portion 1004, may retain the ball 2700 or rounded shape 2700 within the saddle 2702. When a desired angle is achieved, the screw 2704 or other fastener 2704 may be tightened to lock the angle of the pivoting portion 1004 relative to the stationary portion 1002. In the illustrated embodiment, the adjustable-angle multi-unit abutment 1000 and more particularly the pivoting portion 1004 forms an opening 2708 into which a prosthetic dental device, such a overdenture, may be snapped or otherwise attached. As can be appreciated, in other embodiments, the pivoting portion 1004 may be designed or modified to connect to other types of prosthetic dental devices. FIG. 27 shows various views of the adjustable-angle multi-unit abutment 1000 in an angled configuration whereas FIG. 28 shows various views of the adjustable-angle multi-unit abutment 1000 in a straight configuration.

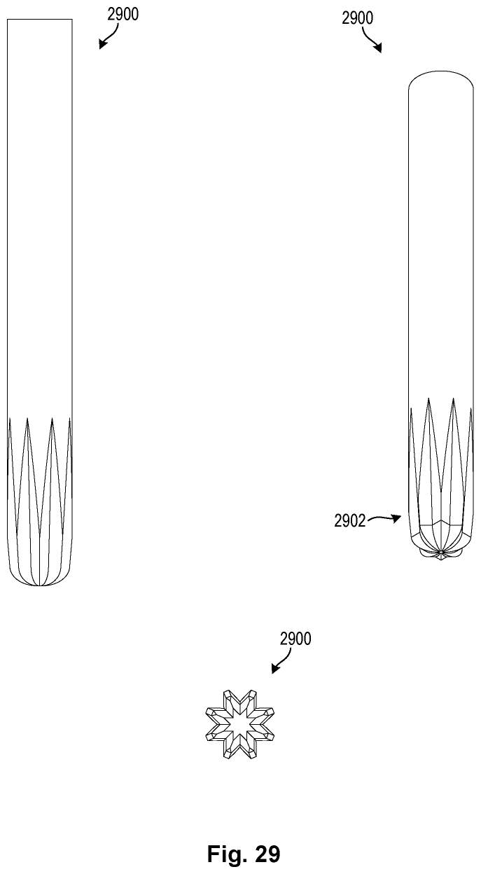

Referring to FIG. 29, in order to securely attach the adjustable-angle multi-unit abutment 1000 within a patient's mouth (such as by connecting the adjustable-angle multi-unit abutment 1000 to an implant), or to fix the angle of the pivoting portion 1004 relative to the stationary portion 1002, or to attach a cylinder 1100 to the pivoting portion 1004 of the adjustable-angle multi-unit abutment 1000, various types of screws may be utilized. In certain cases, visibility and space within a patient's mouth may be limited and/or a certain amount of torque may be required to properly tighten or loosen such screws. Furthermore, the screws that are utilized may be very small (with screw heads on the order of a couple millimeters in diameter). Thus an effective screw head and driver are needed to work in such environments. Ideally, such a screw head and driver may be easy to align with one another in the patient's mouth, and have a design that prevents or minimizes slippage and/or stripping between the screw head and driver.

FIG. 29 shows one embodiment of a driver 2900 that may be used with the screw heads illustrated in FIGS. 22 through 28. A side, perspective, and end view of the driver 2900 are shown. As shown from the end view, in certain embodiments, the driver 2900 may be provided as an eight-pointed star shape. In certain embodiments, this eight-pointed star may be formed with two squares that are rotated by forty-five degrees relative to one another. In certain embodiments, each of these squares may have inwardly bending sides as shown in FIG. 29 such that the angles of the squares is less than ninety degrees (e.g., eighty degrees). In certain embodiments, the ends of the points are slightly rounded or truncated.

Similarly, in certain embodiments, the driver 2900 may be designed to have a morse taper 2902 such that the driver 2900 generates a press fit with the screw head when the driver 2900 is inserted therein. This may enhance the contact and connection between the driver 2900 and the screw head to ideally prevent slipping therebetween or stripping of the screw head. In certain embodiments, this design may enable the driver 2900 to engage the screw head at a slight angle as opposed to needing to be exactly perpendicular to the screw head.

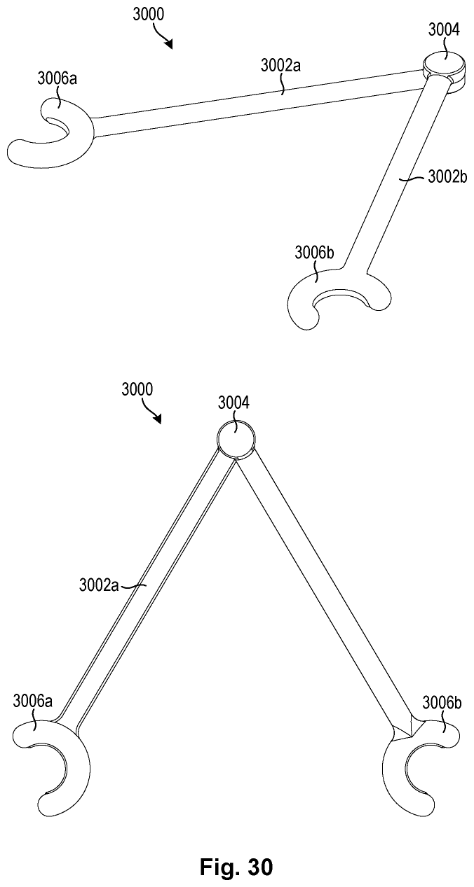

Referring to FIG. 30, when attaching a prosthetic dental device such as a full-arch shell temporary 300 to adjustable-angle multi-unit abutments 1000 within a patient's mouth, the pivoting portions 1004 of the adjustable-angle multi-unit abutments 1000 will ideally be as parallel to one another as possible. To achieve this, the angles of the pivoting portions 1004 of the adjustable-angle multi-unit abutments 1000 may be adjusted accordingly. In order to ensure that each of the pivoting portions 1004 are parallel to one another, a tool may be provided. In certain embodiments, such a tool may be utilized before tightening the set screw 1010 of each of the adjustable-angle multi-unit abutments 1000 within a patient's mouth to ensure that the pivoting portions 1004 are parallel, or to verify that the pivoting portions 1004 are parallel to one another after tightening the set screws 1010. One example of such a tool (hereinafter referred to as a “paralleling jig”) is shown in FIG. 30.

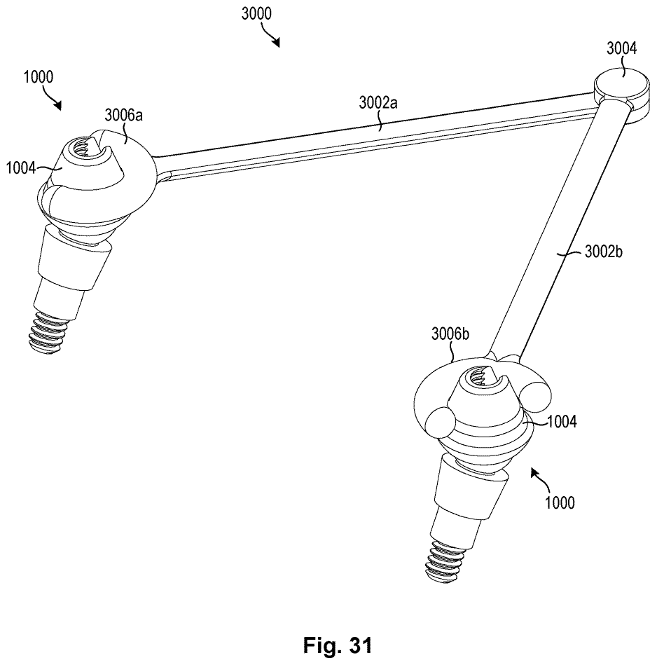

As shown, in certain embodiments, a paralleling jig 3000 in accordance with the invention may include a pair of arms 3002a, 3002b that pivot on a shared axis 3004. Each of the arms 3002a, 3002b may include a clip 3006a, 3006b to grip an adjustable-angle multi-unit abutment 1000, and more specifically a pivoting portion 1004 of an adjustable-angle multi-unit abutment 1000. In certain embodiments, each of the clips 3006a, 3006b is configured to engage a circumferential groove 200 on the adjustable-angle multi-unit abutments 1000. Each of the clips 3006a, 3006b are parallel to one another and may move in the same plane or planes that are parallel to one another. The axis 3004 may enable the clips 3006a, 3006b to engage adjustable-angle multi-unit abutments 1000 that are different distances from one another. Because the clips 3006a, 3006b are parallel to one another, the clips 3006a, 3006b will parallelize the pivoting portions 1004 of the adjustable-angle multi-unit abutments 1000 to which they are connected, as shown in FIG. 31. Once parallel, the set screws 1010 of the adjustable-angle multi-unit abutments 1000 may be tightened to fix the angle of the pivoting portions 1004.

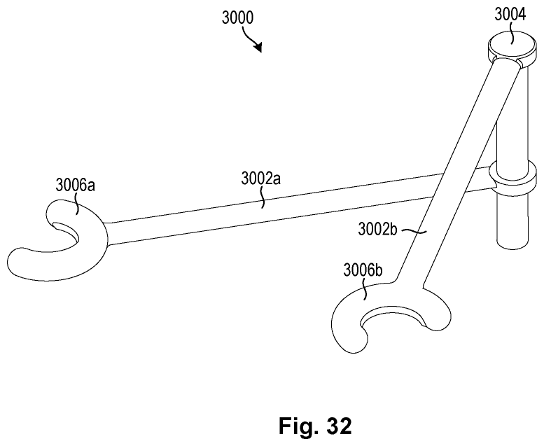

Referring to FIG. 32, in certain cases, it may be advantageous to parallelize the pivoting portions 1004 of adjustable-angle multi-unit abutments 1000 even in cases where the adjustable-angle multi-unit abutments 1000 are at difference heights or levels. In such cases, a paralleling jig 3000 may be modified to parallelize pivoting portions 1004 at different heights or levels. For example, in certain embodiments, the arms 3002a, 3002b of the paralleling jig 3000 may be configured to slide with respect to one another along a shared axis 3004. This may enable the clips 3006a, 3006b to remain parallel to one another even while moving or pivoting in different parallel planes. In certain embodiments, markers may be provided on the shared axis 3004 to enable a distance between the arms 3002a, 3002b to be set to a desired amount.

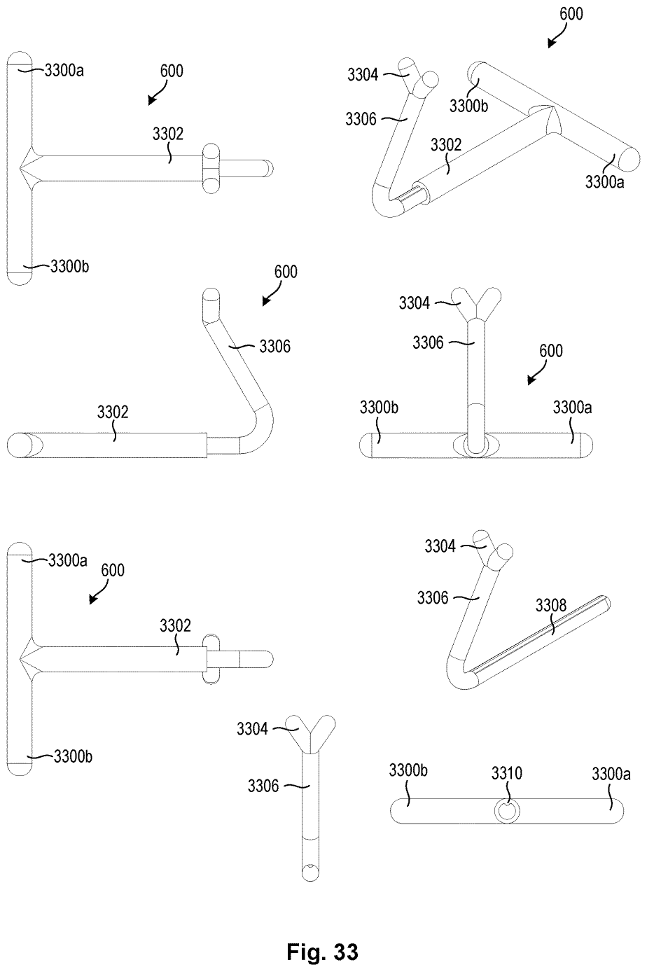

Referring to FIG. 33, as previously described in association with FIG. 6, a measuring tool 600 (referred to herein as a “hamulometer 600”) may be utilized to determine dimensions of a patient's mouth and thus determine what size full-arch shell temporary 300 or other prosthetic dental device a patient may need.

FIG. 33 shows various views of another embodiment of a hamulometer 600 in accordance with the invention. As shown, the hamulometer 600 may be provided in a “T” shape. In certain embodiments, markings (not shown) may be provided at the ends 3300 of the “T” shape. These marking 602 may assist a dentist in measuring side-to-side dimensions of a patient's mouth, such as a distance between the hamular notches of a patient. By contrast, a perpendicular extension 3302 of the hamulometer 600 may be used to measure a patient's jaw or teeth from front to back. This may be accomplished by resting a saddle 3304 of the perpendicular extension 3302 on a nasal spine of the patient. In certain embodiments, a vertical riser 3306 upwardly extends the saddle 3304 from the perpendicular extension 3302 in order to enable contact with the nasal spine.

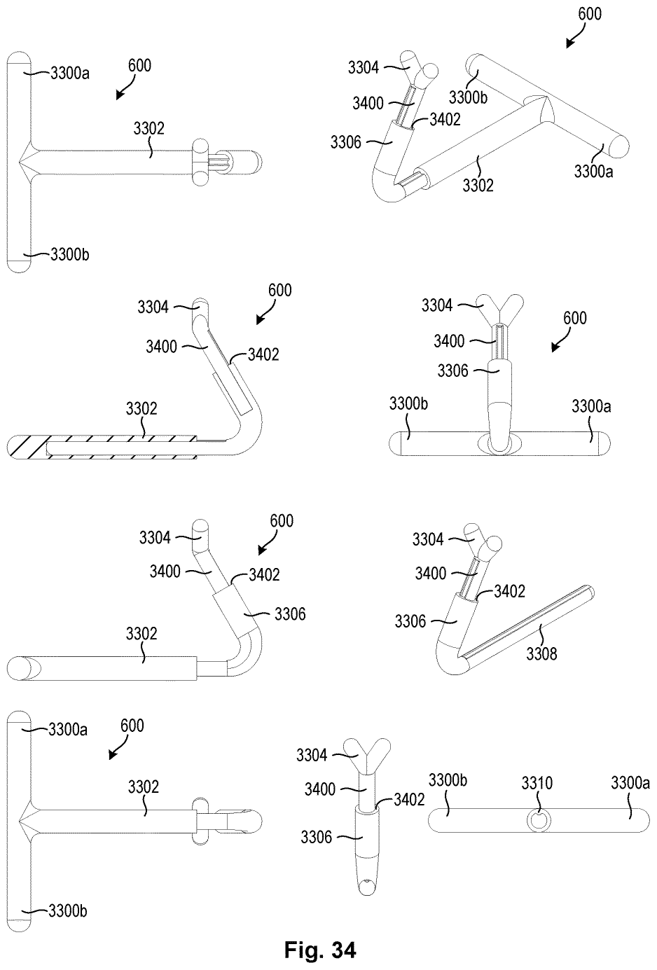

In certain embodiments, a length of the perpendicular extension 3302 is extendable to accommodate different front-to-back dimensions of a patient's mouth. This may be accomplished using a slider 3308 that slides in and out of a channel 3310. Similarly, in certain embodiments, the vertical riser 3306 may also be designed to be extendable, as shown in FIG. 34. FIG. 34 shows a hamulometer 600 that is similar to that illustrated in FIG. 33, except that the vertical riser 3306 is extendable to further accommodate mouths of different dimensions. This may also be accomplished using a slider 3400 that slides in and out of a channel 3402. In certain embodiments, markings are provided on the sliders 3308, 3400 to indicate how far the sliders are extended form their respective channels 3310, 3402 and/or to indicate dimensions of a patient's mouth into which the hamulometer 600 is being used to measure. These measurements may be used to select or design a properly sized prosthetic dental device such as a properly sized full-arch shell temporary 300 for placement in the patient's mouth.

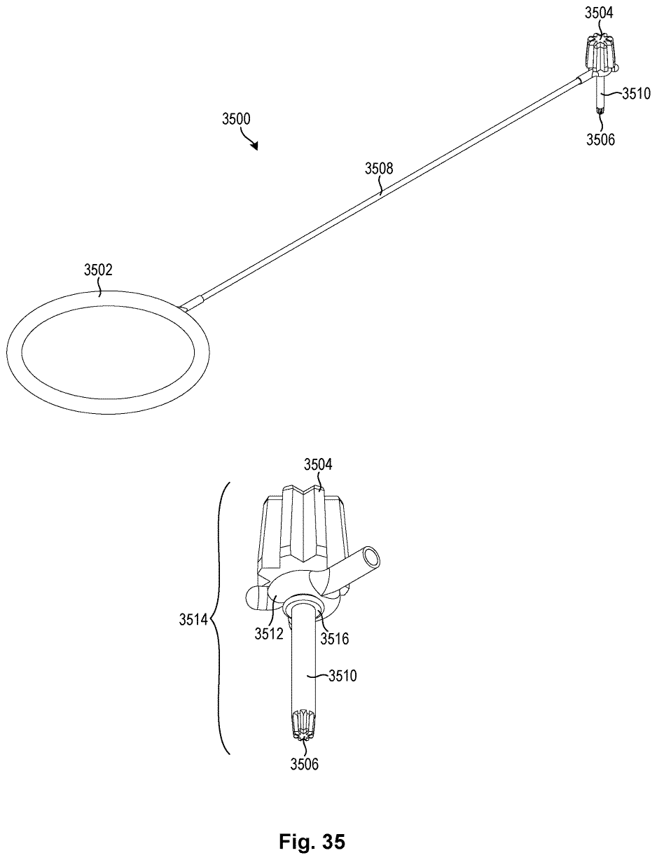

Referring to FIG. 35, in certain embodiments, a driver such as that described in association with FIG. 29 may be incorporated into a tool 3500 that is specifically tailored to dentistry and more specifically to installing/removing the apparatus (e.g., the adjustable-angle multi-unit abutment 1000, the cylinder 1100, the full-arch shell temporary 300, etc.) disclosed herein. Ideally such a tool 3500 will enable a dentist to operate in a patient's mouth while taking into account the environment and space constraints therein. FIG. 35 shows one embodiment of such a tool 3500.

As shown in FIG. 35, in certain embodiments, a tool 3500 in accordance with the invention includes a driver tip 3506 that may generally conform to that described in association with FIG. 29. This driver tip 3506 may be coupled to a shaft 3510 which may in turn be operably coupled to a spline 3504. In certain embodiments, the driver tip 3506 and the spline 3504 have similar geometry but are simply different sizes, with the spline 3504 being significantly larger than the driver tip 3506. The spline 3504 may be rigidly coupled to the driver tip 3506 and thus rotation of the spline 3504 may cause rotation of the driver tip 3506.

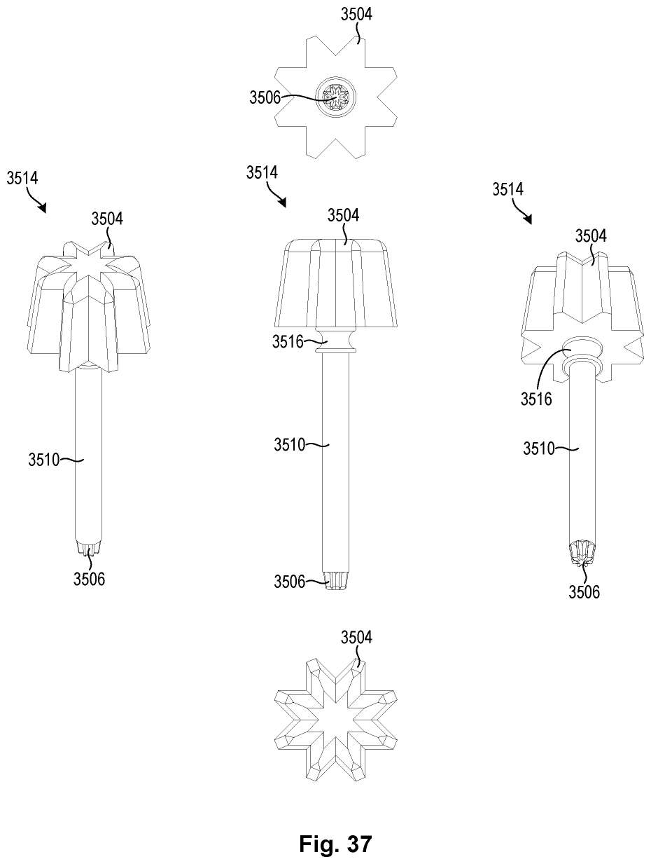

The spline 3504, shaft 3510, and driver tip 3506 together may form a driver component 3514 that may be inserted into a patient's mouth to turn screws used in association with the adjustable-angle multi-unit abutment 1000, cylinder 1100, full-arch shell temporary 300, or other prosthetic dental devices. Because this is a small component 3514, the potential exists for mishandling and/or dropping the driver component 3514 in the patient's mouth during use, with the potential that it may even be inadvertently swallowed by the patient. To prevent this from occurring, in certain embodiments, a tether 3508 may be coupled to the driver component 3514. This tether 3508 may, in certain embodiments, be coupled to a ring 3502 that a dentist may keep on a finger, such as the index finger. In the event the driver component 3514 falls or is mishandled in the patient's mouth, the driver component 3514 may be easily retrieved with the tether 3508 and ring 3502 and thereby avoid swallowing or other issues with the patient. In certain embodiments, the tether 3508 is coupled to a removable clip 3512 that grips the driver component 3514, such as a sleeve 3516 of the driver component 3514. FIG. 37 shows various enlarged views of the driver component 3514 from different viewing angles.

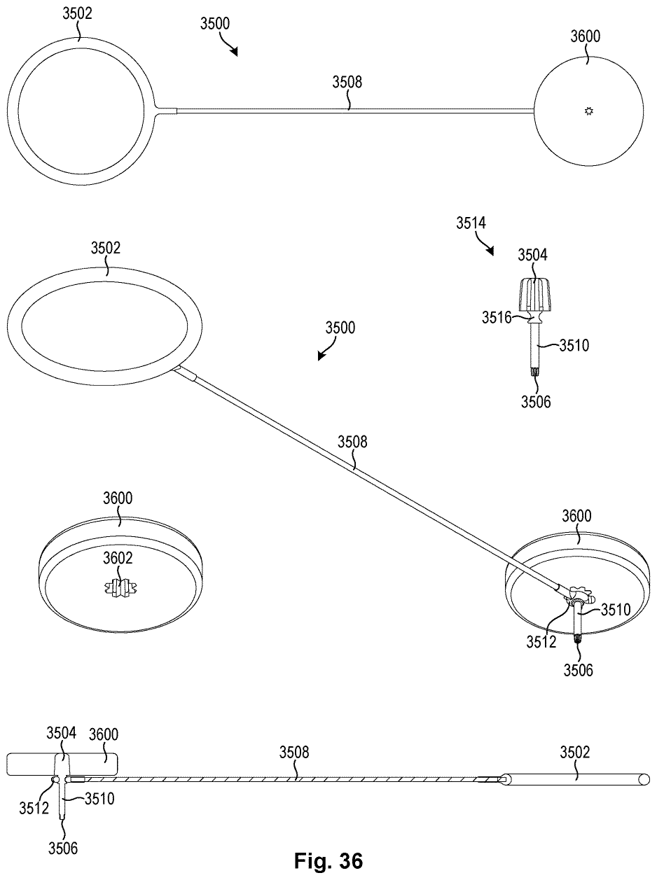

Referring to FIG. 36, in certain embodiments, a finger wheel 3600 may be provided with the tool 3500 to facilitate turning and applying torque to the driver component 3514. This finger wheel 3600 may include a socket 3602 to receive and engage with the spline 3504 of the driver component 3514. The finger wheel 3600 may in certain embodiments be textured or treated to facilitate gripping and turning by a dentist. In certain embodiments, the finger wheel 3600 may be configured with a ratcheting mechanism to enable turning and/or applying torque in one direction only. This ratcheting mechanism may be reversible to turn or apply torque in the opposite direction. Similarly, in certain embodiments, a torquing mechanism may be incorporated into the finger wheel 3600 to enable a dentist to apply a desired amount of torque without over torquing the driver component 3514. In certain embodiments, the finger wheel 3600 includes functionality to enable an amount of torque to be set.

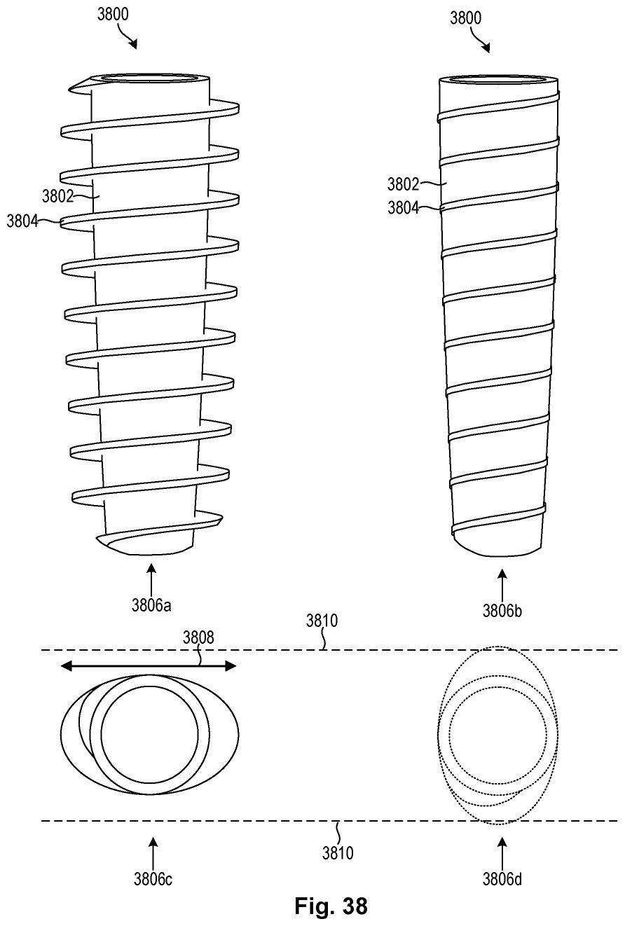

Referring to FIG. 38, when a patient loses teeth, the bone around the teeth often shrinks over time and results in bone loss. These are often the very same patients that eventually need or desire dental implants so that a prosthetic dental device (e.g., full-arch prosthetic teeth, bridge, crown, etc.) can be secured within the patient's mouth. This creates a challenge for the dentist to find an area or location with sufficient bone to which to secure a dental implant. It would be an advance in the art to provide a dental implant that provides the stability of a wider implant but may be used in narrower sections of bone caused by bone loss. One embodiment of such a dental implant 3800 is shown in FIG. 38.

In certain embodiments, a dental implant 3800 in accordance with the invention may include a shaft 3802 and threads 3804 around the shaft 3802. In certain embodiments, the threads 3804 may extend away from the shaft 3802 some designated distance (referred to as “thread depth”) on two opposing sides of the shaft 3802 while being substantially flush with the shaft 3802 on the two opposing sides that are offset by ninety degrees. A first view 3806a and a second view 3806b of the dental implant 3800 are shown in FIG. 38, with the second view 3806b being rotated by ninety degrees relative to the first view 3806a. In certain embodiments, when looking from a top of the dental implant 3800, the shaft 3802 may appear circular while the threads may appear to be in an oval or other flattened shape.

A dental implant 3800 such as that illustrated in FIG. 38 may enable the dental implant 3800 to be placed in bone such that a long dimension 3808 of the threads runs substantially parallel to the bone 3810 (with the bone represented by the area between the dotted lines), as shown by top view 3806c. This same dental implant 3800 may be too wide for the bone 3810 if the long dimension 3808 were placed perpendicular to the bone 3810, as shown by top view 3806d. Thus, the illustrated design may enable a larger dental implant 3800 to be placed in the bone than would otherwise be possible with a dental implant with conventional circular threads. Because a patient may have more bone from front to back than from side to side within the patient's mouth, the illustrated dental implant 3800 may have significant utility when placing implants. In certain embodiments, in order to assist a dentist in correctly orienting the dental implant 3800, a marker may be placed on the dental implant 3800, such as on a top of the dental implant 3800, so that the dentist can verify that a long dimension 3808 of the dental implant 3800 is oriented parallel to the bone.

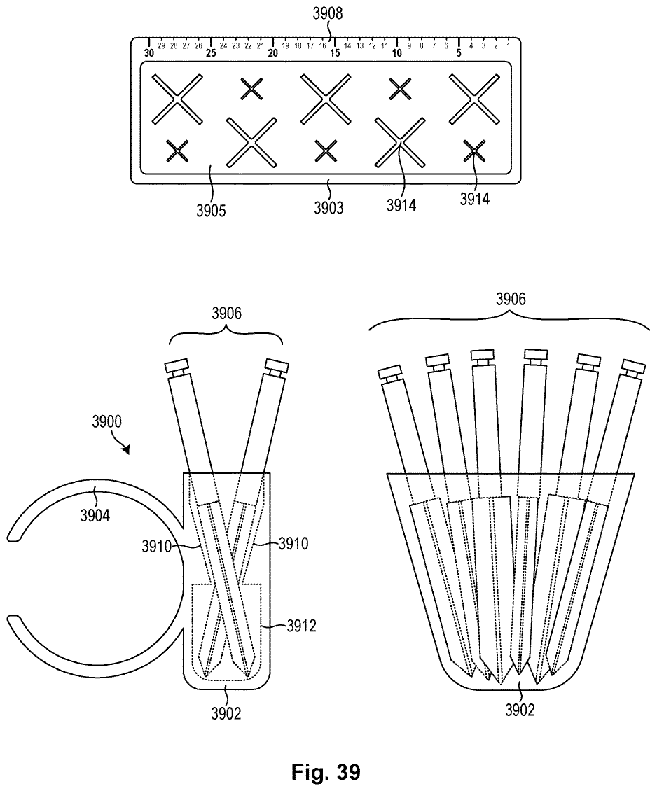

Referring to FIG. 39, dentistry including full-arch dentistry typically requires access to a variety of tools to perform tasks such as placing implants. Any device that improves access to such tools also has the potential to increase the efficiency of practicing dentistry including full-arch dentistry. One example of such a tool 3900 is illustrated in FIG. 39.

In certain embodiments in accordance with the invention, a tool 3900, hereinafter referred to a finger-mounted tool holder 3900, may be provided to hold a selection of tools (e.g., dental burs, drill bits, screw drivers, mirrors, probes, scalers, spoons, etc.). As shown in FIG. 39, the finger-mounted tool holder 3900 includes a sheath 3902 coupled to a ring 3904. The sheath 3902 may be designed to hold a variety of dental tools 3906, such as the illustrated dental drill bits 3906. Because the finger-mounted tool holder 3900 is held on a dentist's finger, the dental tools 3906 are at the dentist's disposal with very little movement or seeking required by the dentist.

In certain embodiments, the sheath 3902 may be designed to keep the dental tools somewhat organized and accessible. For example, in certain embodiments, an interior of the sheath 3902 may contain various angled surfaces 3910 or guides to route the dental tools 3906 in a desired direction. This may assist the dentist in retrieving the dental tools 3906 since ends of the dental tools 3906 may be spread out and distributed for easy handling. Similarly, in certain embodiments, a bottom of the sheath 3902 may taper to further distribute the ends of the dental tools 3906 as well as keep bottom ends of the dental tools 3906 more closely bundled and secure.

As shown in FIG. 39, in certain embodiments, a top side 3903 of the finger-mounted tool holder 3900 may include an opening 3905 to receive the dental tools 3906 as well as a ruler 3908 to measure the dental tools 3906 (e.g., drill bits) when they are retrieved. Dental tools 3906 such as drill bits typically need to be measured each time they are placed in a patient's mouth and thus the ruler 3908 may facilitate such measuring without having to search for a separate measuring instrument. As shown, in certain embodiments, the opening 3905 may include a flexible membrane with apertures 3914 of differing sizes to receive different size dental tools 3906. The apertures 3914 may in certain embodiments include “teeth” that grab onto and retain the dental tools 3906 when they are placed in the sheath 3902.

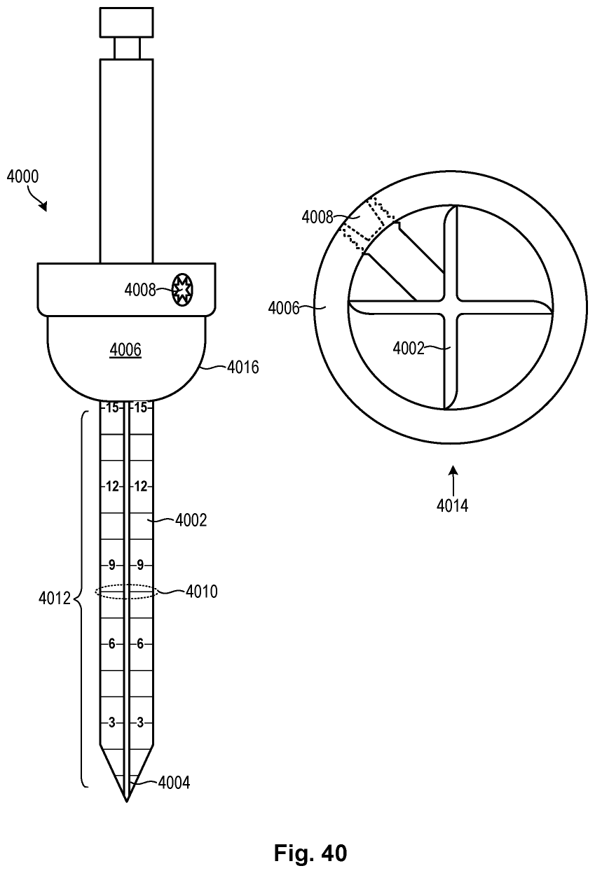

Referring to FIG. 40, in general, to create a hole (also called an “osteotomy”) to receive a dental implant, various tools may be needed. For example, after identifying a location in bone in which to place an implant, the dentist may use a tool to score or create a divot in the patient's tissue and/or bone to identify the location, use a smaller drill bit to form a pilot hole in the patient's bone, and then use a larger drill bit to form a hole that is closer to the size of the implant. After this step, the dentist may utilize a profiling tool to create a profile on a top of the osteotomy to accommodate a prosthetic dental device such as the disclosed adjustable-angle multi-unit abutment 1000 or another abutment. For each step, measurements may be required to ensure that drilling or profiling occurs to a correct depth within the bone. Thus, several steps may be needed to create an osteotomy with the proper characteristics. In would be an advance in the art to provide a tool that could reduce the number of steps to create such an osteotomy.

In certain embodiments in accordance with the invention, a tool 4000 may be provided that significantly reduces a number of steps required to create an osteotomy. As shown, in certain embodiments, the tool 4000 may include a drill bit 4002 or bur 4002 with a pointed end 4004 to score or mark a location for the osteotomy in the patient's bone or tissue. This may taper to a diameter 4010 that is needed to create an osteotomy with sufficient size/diameter to accommodate the dental implant 3800. Cutouts or hollow areas in the sides of the drill bit 4002 or bur 4002 may accommodate bone fragments that are removed while creating the osteotomy. In order to create a desired profile at a top of the osteotomy to accommodate an abutment, a profiler 4006 may be provided on the drill bit 4002 or bur 4002. A lower surface 4016 of the profiler 4006 may include cutting/shaping elements (not shown) to create the desired profile. In certain embodiments, this profiler 4006 is slidable along a length of the drill bit 4002 or bur 4002 with a set screw 4008 to fix a position of the profiler 4006 relative to the drill bit 4002 or bur 4002 once a desired height is determined. A top view 4014 of the profiler 4006, drill bit 4002 or bur 4002, and set screw 4008 are shown in FIG. 40.

To facilitate achieving a desired depth for the osteotomy, markings 4012 may be provided along a length of the drill bit 4002 or bur 4002. The markings may also facilitate proper placement of the profiler 4006, which may also act as a stop when creating the osteotomy. In certain embodiments, the markings 4012 are provided in the form of large numbers representing desired units and/or color coding to make the markings 4012 highly visible and thereby assist a dentist in achieving a proper osteotomy depth. The tool 4000 may be effective to create an osteotomy in a single or smaller number of steps since the scoring, drilling, and profile creation may be performed in a single pass.

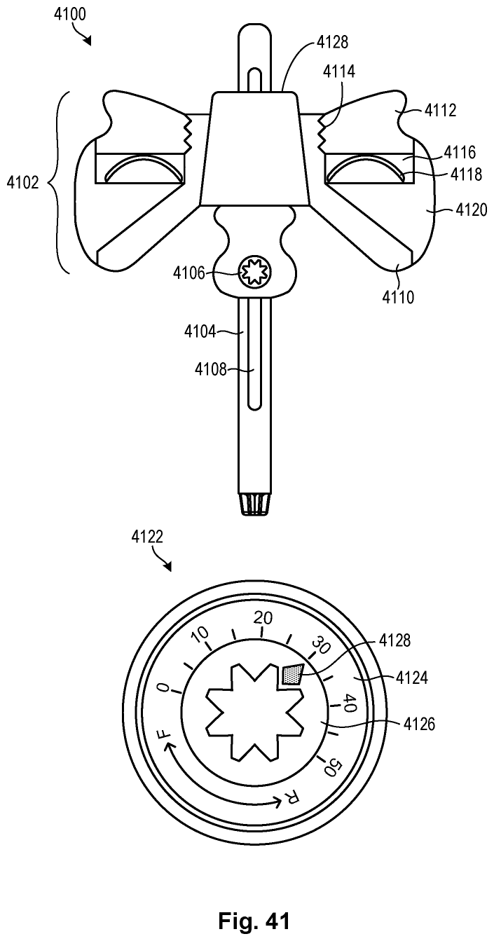

Referring to FIG. 41, as previously described in association with FIG. 36, in certain embodiments, a finger wheel may be used to facilitate turning and applying torque to various types of screw heads. In certain embodiments, the functionality of such a finger wheel may be expanded to include additional features, such as a ratcheting mechanism to facilitate applying torque in only one direction and a torque feature to ensure that each screw is tightened property and with a desired amount of torque, without overtightening. One embodiment of such a tool 4100 or driver 4100 is illustrated in FIG. 41.

As shown, in certain embodiments, the driver 4100 includes a handle 4102 and a driver shaft 4104. In certain embodiments, the driver shaft 4104 may be configured to slide within the handle 4102 to enable a user to establish a height of the handle 4102 relative the driver shaft 4104. In certain embodiments, a set screw 4106 may fix the handle 4102 relative to the driver shaft 4104 once a desired height is achieved, as well as prevent the driver shaft 4104 from rotating relative to the handle 4102. In certain embodiments, the set screw 4106 and/or some cog within the handle 4102 may engage a groove 4108 in the driver shaft 4104 to prevent the driver shaft 4104 from rotating with respect to the handle 4102.

In certain embodiments, the handle 4102 includes a base portion 4110 and a rotating portion 4112 that threads 4114 onto the base portion 4110. A cavity 4116 may be formed between the base portion 4110 and the rotating portion 4112. In certain embodiments, a spring washer 4118 or other resilient element 4118 may be utilized within the cavity to create a spring force between the base portion 4110 and the rotating portion 4112. Turning the rotating portion 4112 relative to the base portion 4110 may compress/decompress this spring washer 4118. As shown in FIG. 41, a torque-application portion 4120 may be compressed between the rotating portion 4112 and the base portion 4110 using the spring washer 4118. In certain embodiments, the spring washer 4118 presses up against a wavy or undulating surface on one or more of the rotating portion 4112 and the torque-application portion 4120.

In order to apply torque to a screw, a user may turn the torque-application portion 4120 which may in turn apply torque to the driver shaft 4104. When a specific amount of torque is reached, the torque-application portion 4120 may slip relative to the rotating portion 4112 and base portion 4110 as a result of the spring washer 4118 slipping on the above-described wavy or undulating surface. The amount of torque required for the torque-application portion 4120 to slip may depend on the amount of compressive force that is applied to the spring washer 4118 within the cavity 4116. In certain embodiments, the amount of compressive force may be adjusted.

FIG. 41 shows one example of a possible top view 4122 of the driver 4100. As shown an outer wheel 4124 (which may be the rotating portion 4112) may rotate relative to an inner hub 4126 (which may be the center hub 4128 of the driver 4100). A user may turn the outer wheel 4124 relative to the inner hub 4126 to establish a torque setting for the driver 4100. A gauge 4128 may indicate the torque setting. Similarly, in certain embodiments, turning or clicking the outer wheel 4124 relative to the inner hub 4126 in one of two directions may cause the ratcheting mechanism of the tool 4100 to transition between a forward and reverse setting.

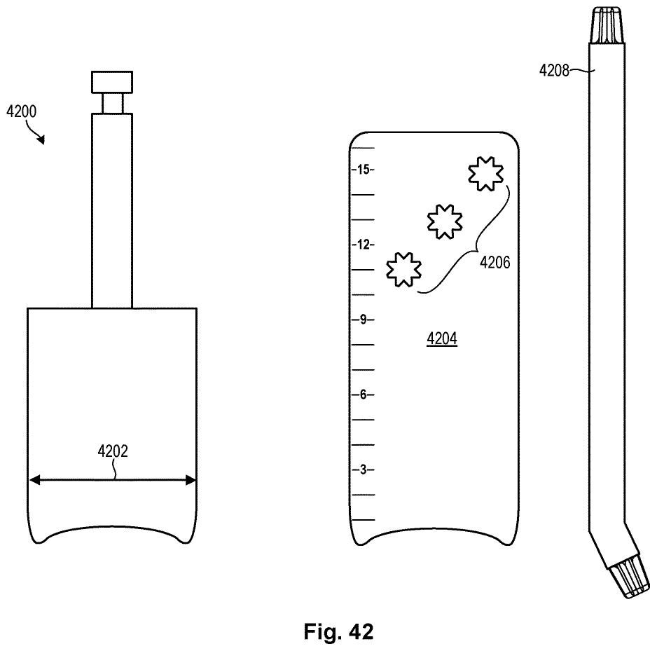

Referring to FIG. 42, when a patient loses molars or teeth on an upper jaw beneath the sinus cavities, within a very short amount of time, the bone may shrink to leave only a thin wafer between the patient's mouth and sinuses. As a result, it may be very difficult to install implants in this very thin and shallow bone, particularly since most dental implants are long and narrow. Thus it would be advance in the art to provide improved implants, as well as apparatus and techniques for installing the same, in the jawbone adjacent to a patient's sinuses.

In certain cases, instead of using long narrow implants in the patient's jawbone adjacent to the sinuses, it may be advantageous to utilize dental implants that are wide and shallow. This may provide significantly more surface contact between the dental implant and the patient's bone. In order to create an osteotomy with greater width, a bur 4200 or drill bit 4200 such as that illustrated in FIG. 42 may be used. This bur 4200 or drill bit 4200 may function in a similar manner to a hole saw where the bone is cored out with a desired diameter 4202, stopping just short of the sinus.

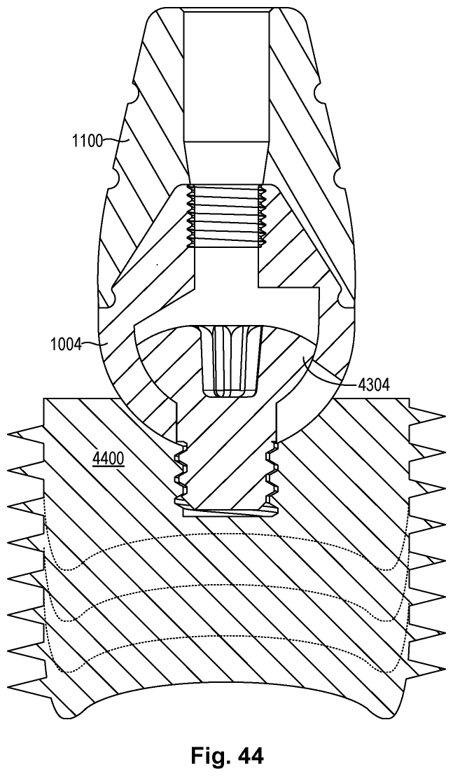

In order to create space for a dental implant that is wide but shallow (one example of which is shown in FIG. 44), an osteotome 4204 may be used. This osteotome 4204 may be hammered or tapped into the osteotomy to break bone at an end of the osteotomy to push it into the sinus cavity to create room for the dental implant. In certain embodiments, the osteotome 4204 that is used is cylindrical and roughly the same diameter as the bur 4200 or drill bit 4200 that was used to create the osteotomy. In certain embodiments, a bottom contour of the osteotome 4204 is roughly equivalent to a bottom contour of the bur 4200 or drill bit 4200 that was used to create the osteotomy. Different diameter osteotomes 4204 may be used for different diameter burs 4200 or drill bits 4200.

The osteotome 4204 may be as simple as a block of steel or other suitable material. In certain embodiments, the osteotome 4204 includes markings along a side thereof to indicate how deep the osteotome 4204 is inserted or driven into the patient's jawbone. Similarly, in certain embodiments, the osteotome 4204 includes various apertures 4206. A removable tool 4208 may be inserted into one of the apertures 4206 in order to place the osteotome 4204 into the osteotomy within the patient's mouth, and/or remove the osteotome 4204 from the patient's mouth once it has been used.

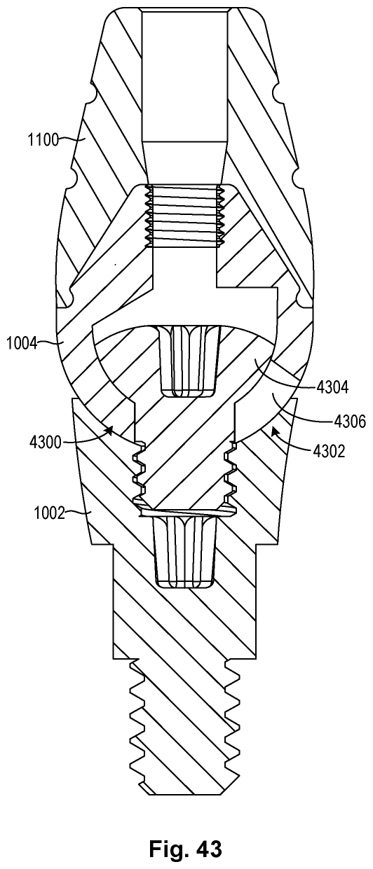

Referring to FIG. 43, in certain embodiments, an adjustable-angle multi-unit abutment 1000 in accordance with the invention may combine various characteristics of the adjustable-angle multi-unit abutments 1000 disclosed in FIGS. 25, 27, and 28. Instead of using a pivoting portion 1004 that pivots on a ball portion 1002. The pivoting portion 1004 may have a ball or circular shape 4300 that pivots within a saddle 4302 of a stationary portion 1002. A slot 4306 may be provided in the circular shape 4300 to facilitate pivoting of the pivoting portion 1004. A top of the pivoting portion 1004 may be designed to interface with the cylinder 1100 previously described, thereby allowing the same cylinder 100 to be used with the adjustable-angle multi-unit abutment 1000. Once a desired angle is achieved, a set screw 4304 may be tightened to fix the angle. In general, the adjustable-angle multi-unit abutment 1000 shown in FIG. 43 may be thought of as having an inverted design compared to the design shown in FIG. 25.

FIG. 44 shows the adjustable-angle multi-unit abutment 1000 of FIG. 43 except that the stationary portion 1002 is replaced with a wide but shallow (typically several millimeters of depth) dental implant 4400. Such a dental implant 4400 may be used advantageously in areas with thin bone, such as the bone between the upper jaw and the sinuses, as was described in association with FIG. 42. The dotted lines on the dental implant 4400 indicate various potential heights in which the dental implant 4400 may be provided. In this embodiment, the pivoting portion 1004 interfaces directly with the dental implant 4400 as opposed to interfacing with a stationary portion 1002 that is then threaded into a dental implant.

While various embodiments of the present disclosure have been described above, it should be understood that they have been presented by way of example only and not limitation. It will be apparent to persons skilled in the relevant art that various changes in form and detail can be made therein without departing from the spirit and scope of the disclosure. Thus, the breadth and scope of the present disclosure should not be limited by any of the above-described exemplary embodiments, but should be defined only in accordance with the following claims and their equivalents. The foregoing description has been presented for the purposes of illustration and description. It is not intended to be exhaustive or to limit the disclosure to the precise form disclosed. Many modifications and variations are possible in light of the above teachings. Further, it should be noted that any or all of the aforementioned alternate implementations may be used in any combination desired to form additional hybrid implementations of the disclosure.

1. A glove-donning apparatus comprising:
a support structure;
a first ring and a second ring attached to the support structure, each ring defining an opening to enable a user's hand to pass through the ring;
wherein each ring is configured to support an opening of an elastic glove stretched thereover; and
wherein insertion of a user's hands into the gloves and movement of the user's hands through the rings causes the gloves to disengage from the rings and contract around the user's hands.

2. The apparatus of claim 1, wherein each ring is non-continuous and extends circumferentially around a majority of a circle.

3. The apparatus of claim 1, wherein the support structure comprises a countertop.

4. The apparatus of claim 1, wherein the rings are attached to the support structure using fasteners.

5. The apparatus of claim 1, wherein the openings of the gloves are stretched across the rings prior to use.

6. The apparatus of claim 1, wherein the openings of the gloves are stretched across the rings with thumbs of the gloves directed toward one another.

7. The apparatus of claim 1, wherein the rings are positioned to receive both hands of the user simultaneously.

8. The apparatus of claim 1, wherein the gloves are configured to travel downward and disengage from the rings as the user's hands are inserted into corresponding fingers of the gloves.

9. The apparatus of claim 1, wherein the gloves contract around the user's hands after disengaging from the rings.

10. The apparatus of claim 1, wherein the apparatus is configured such that the user does not handle the gloves while donning the gloves.

11. A method of donning gloves comprising:
stretching openings of a first glove and a second glove over a first ring and a second ring attached to a support structure, each ring defining an opening to enable a user's hand to pass through the ring;
inserting a user's hands into the gloves; and
moving the user's hands through the rings such that the gloves disengage from the rings and contract around the user's hands.

12. The method of claim 11, wherein each ring is non-continuous and extends circumferentially around a majority of a circle.

13. The method of claim 11, further comprising attaching the rings to the support structure using fasteners.

14. The method of claim 11, wherein the openings of the gloves are stretched across the rings prior to inserting the user's hands into the gloves.

15. The method of claim 11, wherein the openings of the gloves are stretched across the rings with thumbs of the gloves directed toward one another.

16. The method of claim 11, wherein the rings are positioned to receive both hands of the user simultaneously, and the method further comprises inserting both hands into the gloves simultaneously.

17. The method of claim 11, wherein the user inserts fingers of the hands into corresponding fingers of the gloves prior to the gloves disengaging from the rings.

18. The method of claim 11, wherein the gloves are configured to travel downward and disengage from the rings as the user's hands are moved through the rings.

19. The method of claim 11, wherein the gloves contract around the user's hands after the gloves disengage from the rings.

20. The method of claim 11, wherein the method is performed by a dentist or other healthcare worker.
